(19) United States
(12) Patent Application Publication
(10) Pub. No.: US 2023/0055441 A1
(43) Pub. Date: Feb. 23, 2023

(54) PLASMIN-RESISTANT PEPTIDES FOR TREATING STROKE AND RELATED CONDITIONS

(71) Applicant: NONO INC., TORONTO (CA)

(72) Inventors: Michael Tymianksi, Toronto (CA); Jonathan David Garman, Thornhill (CA); Diana Mayor, Toronto (CA)

(21) Appl. No.: 17/791,711

(22) PCT Filed: Jan. 8, 2021
(86) PCT No.: PCT/IB2021/050135

§ 371 Date: Jul. 8, 2022

Related U.S. Application Data
(60) Provisional application No. 62/959,091, filed on Jan. 9, 2020.

(51) Int. Cl. C07K 14/705; C07K 14/47; C07K 7/06; A61P 9/10

(52) U.S. Cl. CPC C07K 14/70571; C07K 14/47; C07K 7/06; A61P 9/10; C07K 2319/10

(57) ABSTRACT

The invention provides variants of a previously described active agent for treating stroke, Tat-NR2B9c, in which target binding characteristics are retained by inclusion of L-amino acids at the C-terminus and plasmin-resistance is conferred by inclusion of D-amino acids elsewhere. An exemplary agent has the sequence ygrkkrrqrrrklssIETDV (SEQ ID NO:62). The resulting active agents have several advantages including administration at the same time as thrombolytic agents without significant loss of activity due to plasmin digestion. The resulting agents are also more suitable for administration by alternative routes to intravenous infusion, such as subcutaneous, intranasal and intramuscular, and for multi-dosing regimes for treatment of chronic conditions.

### CROSS-REFERENCE TO RELATED APPLICATION

The present application claims priority from U.S. 62/959,091 filed Jan. 9, 2020, which is incorporated by reference in its entirety for all purposes.

### SEQUENCE LISTING

The present application includes sequences in a txt filed named 695323WO of 20 kbytes, created Jan. 7, 2021, which is incorporated by reference.

### BACKGROUND

Tat-NR2B9c (also known as NA-1) is an agent that inhibits PSD-95, thus disrupting binding to N-methyl-D-aspartate receptors (NMDARs) and neuronal nitric oxide synthases (nNOS) and reducing excitotoxicity induced by cerebral ischemia. Treatment reduces infarction size and functional deficits in models of cerebral injury and neurodegenerative diseases. Tat-NR2B9c has undergone a successful phase II trial (see WO 2010144721 and Aarts et al., Science 298, 846-850 (2002), Hill et al., Lancet Neurol. 11:942-950 (2012)) and a successful Phase 3 trial (Hill et al, Lancet 395:878-887 (2020)).

Except for glycine, all standard α-amino acids can exist in either of two optical isomers, which are mirror image of one other called L- and D-amino acids. Proteins and most naturally occurring peptides are formed entirely of amino acids in the L-configuration. D-amino acids have been detected in a only few natural peptides. These D-amino acids form when L-amino acids undergo posttranslational alterations. Because of the rarity of D-amino acids in nature, they are generally not recognized by L-proteins at least to the same extent as L-amino acids. Simply replacing L for D amino acids is generally ineffective in creating mimetics of a parent molecule because it alters side chain orientations with respect to target sites. Replacing L or D amino acids and reversing the order of amino acids results in side-chain topology similar to the parent molecule but with inverted amide peptide bonds, which adapt left-hand helices, whereas L peptides adapt right-handed helices. Thus, target binding can still be lost or altered.

### SUMMARY OF THE CLAIMED INVENTION

The invention provides an active agent comprising an internalization peptide linked to an inhibitor peptide, which inhibits PSD-95 binding to NOS and/or NMDAR2B, wherein the internalization peptide has an amino acid sequence comprising YGRKKRRQRRR (SEQ ID NO:1) and the inhibitor peptide has a sequence comprising KLSSIESDV (SEQ ID NO:2), or a variant thereof with up to five substitutions or deletions total in the internalization peptide and inhibitor peptide, wherein at least the four C-terminal amino acids of the inhibitor peptide are L-amino acids, and a contiguous segment of amino acids including all of the R and K residues are D-amino acids. Optionally, the residue immediately C-terminal to the most C-terminal R or K residue is also a D-residue. Optionally, the C-terminal of the internalization peptide is linked to the N-terminus of the inhibitor peptide as a fusion peptide. Optionally, the inhibitor peptide comprises [E/D/N/Q]-[S/T]-[D/E/Q/N]-[V/L] (SEQ ID NO:3) at the C-terminus. Optionally, the inhibitor peptide comprises I-E-[S/T]-D-V (SEQ ID NO:4) at the C-terminus. Optionally, the inhibitor peptides comprises IESDV (SEQ ID NO:5) at the C-terminus.

Optionally each of the five C-terminal amino acids of the inhibitor peptide are L-amino acids. Optionally, each other residue of the active agent is a D-amino acid. Optionally, the active agent has the amino acid sequence ygrkkrrqrrrklsslESDV (SEQ ID NO:6), ygrkkrrqrrrksslESDV (SEQ ID NO:7), ygrkkrrqrrrkslESDV (SEQ ID NO:8), or ygrkkrrqrrrklESDV (SEQ ID NO:9). Optionally, the active agent has the amino acid sequences ygrkkrrqrrrklsslESDV (SEQ ID NO:6), wherein the lower case letters are D-amino acids and the upper case letters are L-amino acids.

Optionally, the active agent has enhanced stability in plasma compared with Tat-NR2B9c. Optionally, the active agent has enhanced plasmin resistance compared with Tat-NR2B9c. Optionally, the active agent has a binding affinity for PSD-95 within 2- fold of Tat-NR2B9c. Optionally, the active agent has an IC50 for inhibiting PSD-95 binding to NMDAR2B within 2-fold of Tat-NR2B9c.

Optionally, the active agent is a chloride salt.

The invention further provides a formulation of any of the active agents further comprising histidine and trehalose.

The invention further provides a formulation of any of the active agents further comprising a phosphate buffer.

The invention further provides coformulation comprising any of the active agents and an anti-inflammatory agent. Optionally, the anti-inflammatory is a mast cell degranulation inhibitor or antihistamine.

The invention further provides a co-formulation comprising any of the active agents and a thrombolytic agent.

The invention further provides a method of treating a subject having or at risk of a condition selected from stroke, cerebral ischemia, traumatic injury to the CNS, subarachnoid hemorrhage, pain, anxiety, epilepsy, comprising administering an effective regime of any of the active agents to the subject.

The invention further provides a method of treating ischemic stroke in a subject having or at risk of stroke, comprising administering an effective regime of an active agent to the subject, wherein the subject is co-administered a thrombolytic agent, wherein the active agent comprises an internalization peptide linked to an inhibitor peptide, which inhibits PSD-95 binding to NOS and/or NMDAR2B, wherein at least the four C-terminal amino acids of the inhibitor peptide are L-amino acids, and at least one of the remaining amino acids of the active agent is a D-amino acid, wherein the active agent and thrombolytic agent are administered sufficiently proximate in time that cleavage of the active agent induced by the thrombolytic agent is reduced by the inclusion of the at least one D-amino acid. Optionally, the internalization peptide is linked at its N-terminus to the C-terminus of the inhibitor peptide as a fusion protein. Optionally, the inhibitor peptide comprises [E/D/N/Q]-[S/T]-[D/E/Q/N]-[V/L](SEQ ID NO:3) as the last four residues. Optionally, the inhibitor peptide comprises [I]-[E/D/N/Q]-[S/T]-[D/E/Q/N]-[V/L] (SEQ ID NO:10) as the last five residues, each of which is an L amino acid. Optionally, the internalization peptide is a tat peptide. Optionally, at least 8 residues of the tat peptide are D-amino acids. Optionally, each residue of the tat peptide is a D-amino acid. Optionally, the internalization peptide comprises GRKKRRQRRR (SEQ ID NO:11) linked at its N-terminus to KLSSIESDV (SEQ ID NO:2) or KLSSIETDV (SEQ ID NO:12) as the inhibitor peptide forming a fusion protein. Optionally, the active agent comprises a contiguous segment of D-residues including each of the K and R residues. Optionally, the active agent comprises ygrkkrrqrrrklsslESDV (SEQ ID NO:6), wherein lower case letters represent D-amino acids and upper case letters are L-amino acids. Optionally, the thrombolytic agent is administered within a window of 60, 30 or 15 minutes before the active agent. Optionally, the active agent and thrombolytic agent are administered at the same time.

The invention further provides a method of delivering an active agent to a subject in need thereof, comprising administering the active agent as defined in any preceding claim by a nonintravenous route, wherein the active agent is delivered to the plasma at a therapeutic level. Optionally, the active agent is administered subcutaneously. Optionally, the active agent is administered intramuscularly. Optionally, the active agent is administered intranasally or intrapulmonarily. Optionally, the dose is greater than 3 mg/kg. Optionally, the dose is greater than 10 mg/kg. Optionally, the dose is greater than 20 mg/kg. Optionally, the dose is below 10 mg/kg and the variant is administered without co-administration of a mast cell degranulating inhibitor or anti-histamine. Optionally, the dose is above 10 mg/kg and the variant is administered. Optionally, the subject has or is at risk of a condition selected from stroke, cerebral ischemia, traumatic injury to the CNS, pain, anxiety, epilepsy, subarachnoid hemorrhage, Alzheimer's disease or Parkinson's disease.

### BRIEF DESCRIPTIONS OF THE FIGURES

FIG. 1. Plasmin cleavage sites on NA-1 (SEQ ID NO:58).

FIG. 2 NA-1 content in rat plasma is significantly reduced when given simultaneously with rt-PA.

FIG. 3 NA-1 content in human plasma is significantly reduced when given simultaneously with rt-PA.

FIG. 4 NA-1 Cmax and AUC is significantly reduced when administered simultaneously with rt-PA (5.4 mg/kg).

FIG. 5: D-Tat-L-NR2B9c demonstrate superior stability in rat plasma in the presence of rt-PA when compared to NA-1.

FIG. 6: D-Tat-L-NR2B9c is resistant to proteolysis during rt-PA infusion in human plasma.

FIG. 7 NA-1 content in human plasma is reduced when given simultaneously with TNK, but D-Tat-L-NR2B9c content is preserved.

FIG. 8 NA-1 content in rat plasma is reduced when given simultaneously with TNK, but D-Tat-L-NR2B9c content is preserved.

FIG. 9: D-Tat-L-NR2B9c is resistant to plasmin cleavage in PBS medium.

FIG. 10: Results: D-Tat-L-NR2B9c dissociates pre-formed NR2B:PSD95 complexes in rat brain lysates.

FIG. 11: D-Tat-L-NR2B9c and D-Tat-L-IESDV (SEQ ID NO:6) effectively bind the target protein PSD95-PDZ2.

FIG. 12: Result: NA-1 and D-Tat-L-NR2B9c have a high binding affinity for PSD95-PDZ2 domain.

FIG. 13: Subcutaneous NA-1 achieved similar plasma exposure relative to IV NA-1.

FIG. 14: Subcutaneous NA-3 achieved higher plasma concentration and a greater plasma exposure relative to subcutaneous NA-1.

FIG. 15A (Table) FIG. 15B (charts): Subcutaneous NA-3 achieved greater plasma exposure relative to SQ NA-1.

FIG. 16: pulmonary instillation of D-NA-1 and NA-3 achieved higher plasma concentration and a greater plasma exposure relative to Intrapulmonary NA-1.

FIG. 17: Lack of significant histamine release after subcutaneous administration of NA-3 at a dose of 8.3 mg/kg or 2.8 mg/kg dose.

FIG. 18: No significant histamine release after intravenous administration of the co-formulation of D-Tat-L-NR2B9c (7.6 mg/kg) and lodoxamide (0.6 mg/kg).

FIG. 19: Intravenous administration of D-Tat-L-NR2B9c and lodoxamide 1 hour after stroke onset reduced infarct volume and hemispheric swelling in animals subjected to an eMCAo model.

FIG. 20: D-Tat-L-NR2B9c and lodoxamide administration resulted in an improved neurological outcome 24 hours after stroke onset.

FIG. 21: Effect of subcutaneous NA-3 and nerinetide on infarct volume

FIG. 22: nerinetide and NA-3 plasma concentrations at 15 minutes post subcutaneous dose

FIG. 23: Subcutaneous NA-3 at 25 mg/kg resulted in a greater Cmax and AUC than nerinetide IV infusion

FIG. 24: NA-3 pharmacokinetic profile after subcutaneous administration.

### DEFINITIONS

A “pharmaceutical formulation” or composition is a preparation that permits an active agent to be effective, and lacks additional components which are toxic to the subjects to which the formulation would be administered.

Use of upper case one letter amino acid codes can refer to either D or L amino acids unless the context indicates otherwise. Lower case single letter codes are used to indicate D amino acids. Glycine does not have D and L forms and thus can be represented in either upper or lower case interchangeably.

Numeric values such as concentrations or pH's are given within a tolerance reflecting the accuracy with which the value can be measured. Unless the context requires otherwise, fractional values are rounded to the nearest integer. Unless the context requires otherwise, recitation of a range of values means that any integer or subrange within the range can be used.

The terms “disease” and “condition” are used synonymously to indicate any disruption or interruption of normal structure or function in a subject.

Indicated dosages should be understood as including the margin of error inherent in the accuracy with which dosages can be measured in a typical hospital setting

The terms “isolated” or “purified” means that the object species (e.g., a peptide) has been purified from contaminants that are present in a sample, such as a sample obtained from natural sources that contain the object species. If an object species is isolated or purified it is the predominant macromolecular (e.g., polypeptide) species present in a sample (i.e., on a molar basis it is more abundant than any other individual species in the composition), and preferably the object species comprises at least about 50 percent (on a molar basis) of all macromolecular species present. Generally, an isolated, purified or substantially pure composition comprises more than 80 to 90 percent of all macromolecular species present in a composition. Most preferably, the object species is purified to essential homogeneity (i.e., contaminant species cannot be detected in the composition by conventional detection methods), wherein the composition consists essentially of a single macromolecular species. The term isolated or purified does not necessarily exclude the presence of other components intended to act in combination with an isolated species. For example, an internalization peptide can be described as isolated notwithstanding that it is linked to an active peptide.

A “peptidomimetic” refers to a synthetic chemical compound which has substantially the same structural and/or functional characteristics of a peptide consisting of natural amino acids. The peptidomimetic can contain entirely synthetic, non-natural analogues of amino acids, or can be a chimeric molecule of partly natural peptide amino acids and partly non-natural analogs of amino acids. The peptidomimetic can also incorporate any amount of natural amino acid conservative substitutions as long as such substitutions also do not substantially alter the mimetic's structure and/or inhibitory or binding activity. Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. In a peptidomimetic of a chimeric peptide comprising an active peptide and an internalization peptide, either the active moiety or the internalization moiety or both can be a peptidomimetic.

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

Excitotoxicity is the pathological process by which neurons and surrounding cells are damaged and killed by the overactivation of receptors for the excitatory neurotransmitter glutamate, such as the NMDA receptors, e.g., NMDA receptors bearing the NMDAR2B subunit.

The term “subject” includes humans and veterinary animals, such as mammals, as well as laboratory animal models, such as mice or rats used in preclinical studies.

A tat peptide means a peptide comprising or consisting of RKKRRQRRR (SEQ ID NO:13), in which no more than 5 residues are deleted, substituted or inserted within the sequence, which retains the capacity to facilitate uptake of a linked peptide or other agent into cells. Preferably any amino acid changes are conservative substitutions. Preferably, any substitutions, deletions or internal insertions in the aggregate leave the peptide with a net cationic charge, preferably similar to that of the above sequence. Such can be accomplished for example, by not substituting any R or K residues, or retaining the same total of R and K residues. The amino acids of a tat peptide can be derivatized with biotin or similar molecule to reduce an inflammatory response.

Co-administration of a pharmacological agents means that the agents are administered sufficiently close in time for detectable amounts of the agents to present in the plasma simultaneously and/or the agents exert a treatment effect on the same episode of disease or the agents act co-operatively, or synergistically in treating the same episode of disease. For example, an anti-inflammatory agent acts cooperatively with an agent including a tat peptide when the two agents are administered sufficiently proximately in time that the anti-inflammatory agent can inhibit an anti-inflammatory response inducible by the internalization peptide.

Statistically significant refers to a p-value that is <0.05, preferably <0.01 and most preferably <0.001.

An episode of a disease means a period when signs and/or symptoms of the disease are present interspersed by flanked by longer periods in which the signs and/or symptoms or absent or present to a lesser extent.

The term “NMDA receptor,” or “NMDAR,” refers to a membrane associated protein that is known to interact with NMDA including the various subunit forms described below. Such receptors can be human or non-human (e.g., mouse, rat, rabbit, monkey).

Reference to object as comprising a specified feature should be understood as alternatively disclosing the object consisting of or consisting essentially of the specified feature. Likewise reference to an object as consisting of or consisting of a feature should be understood as alternatively disclosing the object comprising or consisting essentially of the feature. Likewise reference to an object as consisting essentially of a feature, should be understood as alternatively disclosing the object consisting of or comprising the feature. Consisting essentially of is used in accordance with convention to refer to the basic and novel features of an invention.

### DETAILED DESCRIPTION

I. General

The invention provides variants of the previously described active agent for treating stroke, Tat-NR2B9c, in which the C-terminal four or five amino acids are L-amino acids and one or more of the remaining amino acids are D-amino acids. The inclusion of D-amino acids inhibits proteolytic degradation of the agent, particularly by plasmin, which is present in the plasma naturally and is induced by administration of thrombolytic agents. The retention of L-amino acids at the C-terminus is sufficient to retain the binding and inhibitory characteristics of Tat-NR2B9c notwithstanding the presence of D-amino acids in some or all of the rest of the molecule. The resulting active agents have several advantages including increased half-life, and resistance to plasmin induced by co-administered or co-formulated thrombolytic agents. The resulting agents are also more suitable for administration by alternative routes to intravenous infusion, such as subcutaneous, intranasal and intramuscular because the longer half-life of the agents can compensate for the longer time required by these routes to develop a therapeutic concentration in the plasma. Administration by such routes allows administration of higher dosages without significant histamine release as well as being more suitable for performance in the field rather than a medical facility. The greater half-life of the active agents of the invention also makes them more suitable for maintaining a therapeutic concentration over a prolonged period of time in a multidosing regime. Such regimes can be useful for promoting recovering from pathological and cognitive deficits resulting from stroke as well as reducing the initial deficits. Multidosing regimes can be also be useful for treating chronic conditions, such as Alzheimer's and Parkinson's disease.

II. Active Agents

Active agents of the invention include a peptide inhibitor specifically binding to PSD-95 (e.g., Stathakism, Genomics 44(1):71-82 (1997)) so as to inhibit its binding to NMDA Receptor 2 subunits including NMDAR2B (e.g., GenBank ID 4099612) and/or NOS (e.g., neuronal or nNOS Swiss-Prot P29475), and an internalization peptide to facilitate passage of the peptide inhibitor across cell membranes and the blood brain barrier. Preferred peptides inhibit the human forms of PSD-95 NMDAR 2B and NOS for use in a human subject. However, inhibition can also be shown from species variants of the proteins. Some peptide inhibitors have an amino acid sequence comprising [E/D/N/Q]-[S/T]-[D/E/Q/N]-[V/L] (SEQ ID NO:3) at their C-terminus. Exemplary peptides comprise: ESDV (SEQ ID NO:14), ESEV (SEQ ID NO:15), ETDV (SEQ ID NO:16), ETAV (SEQ ID NO:17), ETEV (SEQ ID NO:18), DTDV (SEQ ID NO:19), and DTEV (SEQ ID NO:20) as the C-terminal amino acids. Some peptides have an amino acid sequence comprising [I]-[E/D/N/Q]-[S/T]-[D/E/Q/N]-[V/L] (SEQ ID NO:10) at their C-terminus. Exemplary peptides comprise: IESDV (SEQ ID NO:5), IESEV (SEQ ID NO:21), IETDV (SEQ ID NO:22), IETAV (SEQ ID NO:23), IETEV (SEQ ID NO:24), IDTDV (SEQ ID NO:25), and IDTEV (SEQ ID NO:26) as the C-terminal amino acids. Some inhibitor peptides having an amino acid sequence comprising X₁-[T/S]-X₂V (SEQ ID NO:27) at the C-terminus, wherein [T/S] are alternative amino acids, X₁ is selected from among E, Q, and A, or an analogue thereof, X₂ is selected from among A, Q, D, N, N-Me-A, N-Me-Q, N-Me-D, and N-Me-N or an analog thereof (see Bach, J. Med. Chem. 51, 6450-6459 (2008) and WO 2010/004003). Optionally the peptide is N-alkylated in the P3 position (third amino acid from C-terminus, i.e. position occupied by [T/S]). The peptide can be N-alkylated with a cyclohexane or aromatic substituent, and further comprises a spacer group between the substituent and the terminal amino group of the peptide or peptide analogue, wherein the spacer is an alkyl group, preferably selected from among methylene, ethylene, propylene and butylene. The aromatic substituent can be a naphthalen-2-yl moiety or an aromatic ring substituted with one or two halogen and/or alkyl group. Some inhibitor peptides having an amino acid sequence comprising IX₁-[T/S]-X₂V (SEQ ID NO:28) at the C-terminus. Exemplary inhibitor peptides have sequences IESDV (SEQ ID NO:5), IETDV (SEQ ID NO:22), KLSSIESDV (SEQ ID NO:2), and KLSSIETDV (SEQ ID NO:12). Inhibitor peptides usually have 3-25 amino acids (without an internalization peptide), peptide lengths of 5-10 amino acids, and particularly 9 amino acids (also without an internalization peptide) are preferred.

Internalization peptides are a well-known class of relatively short peptides that allow many cellular or viral proteins to traverse membranes. They can also promote passage of linked peptides across cell membranes or the blood brain barrier. Internalization peptides, also known as cell membrane transduction peptides, protein transduction domains, brain shuttles or cell penetrating peptides can have e.g., 5-30 amino acids. Such peptides typically have a cationic charge from an above normal representation (relative to proteins in general) of arginine and/or lysine residues that is believed to facilitate their passage across membranes. Some such peptides have at least 5, 6, 7 or 8 arginine and/or lysine residues. Examples include the antennapedia protein (Bonfanti, Cancer Res. 57, 1442-6 (1997)) (and variants thereof), the tat protein of human immunodeficiency virus, the protein VP22, the product of the UL49 gene of herpes simplex virus type 1, Penetratin, SynB1 and 3, Transportan, Amphipathic, gp41NLS, polyArg, and several plant and bacterial protein toxins, such as ricin, abrin, modeccin, diphtheria toxin, cholera toxin, anthrax toxin, heat labile toxins, and Pseudomonas aeruginosa exotoxin A (ETA). Other examples are described in the following references (Temsamani, Drug Discovery Today, 9(23):1012-1019, 2004; De Coupade, Biochem J., 390:407-418, 2005; Saalik Bioconjugate Chem. 15: 1246-1253, 2004; Zhao, Medicinal Research Reviews 24(1):1-12, 2004; Deshayes, Cellular and Molecular Life Sciences 62:1839-49, 2005); Gao, ACS Chem. Biol. 2011, 6, 484-491, SG3 (RLSGMNEVLSFRWL (SEQ ID NO:29)), Stalmans PLoS ONE 2013, 8(8) e71752, 1-11 and supplement; Figueiredo et al., IUBMB Life 66, 182-194 (2014); Copolovici et al., ACS Nano, 8, 1972-94 (2014); Lukanowski, Biotech J. 8, 918-930 (2013); Stockwell, Chem. Biol. Drug Des. 83, 507-520 (2014); Stanzl et al., Accounts. Chem. Res/46, 2944-2954 (2013); Oller-Salvia et al., Chemical Society Reviews 45: 10.1039/c6cs00076b (2016); Behzad Jafari et al., (2019) Expert Opinion on Drug Delivery, 16:6, 583-605 (2019) (all incorporated by reference). Still other strategies use additional methods or compositions to enhance delivery of cargo molecules such as the PSD-95 inhibitors to the brain (Dong, Theranostics 8(6): 1481-1493 (2018).

A preferred internalization peptide is tat from the HIV virus. A tat peptide reported in previous work comprises or consists of the standard amino acid sequence YGRKKRRQRRR (SEQ ID NO:1) found in HIV Tat protein. RKKRRQRRR (SEQ ID NO:13) and GRKKRRQRRR (SEQ ID NO:11) can also be used. If additional residues flanking such a tat motif are present (beside the pharmacological agent) the residues can be for example natural amino acids flanking this segment from a tat protein, spacer or linker amino acids of a kind typically used to join two peptide domains, e.g., gly (ser)₄ (SEQ ID NO:30), TGEKP (SEQ ID NO:31), GGRRGGGS (SEQ ID NO:32), or LRQRDGERP (SEQ ID NO:33) (see, e.g., Tang et al. (1996), J. Biol. Chem. 271, 15682-15686; Hennecke et al. (1998), Protein Eng. 11, 405-410)), or can be any other amino acids that do not significantly reduce capacity to confer uptake of the variant without the flanking residues. Preferably, the number of flanking amino acids other than an active peptide does not exceed ten on either side of YGRKKRRQRRR (SEQ ID NO:1). However, preferably, no flanking amino acids are present. One suitable tat peptide comprising additional amino acid residues flanking the C-terminus of YGRKKRRQRRR (SEQ ID NO:1) or other inhibitor peptide is YGRKKRRQRRRPQ (SEQ ID NO:34). Other tat peptides that can be used include GRKKRRQRRRPQ (SEQ ID NO:35 and GRKKRRQRRRP (SEQ ID NO:36).

Variants of the above tat peptide having reduced capacity to bind to N-type calcium channels are described by WO2008/109010. Such variants can comprise or consist of an amino acid sequence XGRKKRRQRRR (SEQ ID NO:37), in which X is an amino acid other than Y or can comprise or consist of an amino acid sequence GRKKRRQRRR (SEQ ID NO:11). A preferred tat peptide has the N-terminal Y residue substituted with F. Thus, a tat peptide comprising or consisting of FGRKKRRQRRR (SEQ ID NO:38) is preferred. Another preferred variant tat peptide consists of GRKKRRQRRR (SEQ ID NO:11). Another preferred tat peptide comprises or consists of RRRQRRKKRG (SEQ ID NO:39) or RRRQRRKKRGY (SEQ ID NO:40). Other tat derived peptides that facilitate uptake of a pharmacological agent without inhibiting N-type calcium channels include those presented in Table 1 below.

TABLE 1
X-FGRKKRRQRRR (F-Tat) (SEQ ID NO: 38)
X-GKKKKKQKKK (SEQ ID NO: 41)
X-RKKRRQRRR (SEQ ID NO: 13)
X-GAKKRRQRRR (SEQ ID NO: 42)
X-AKKRRQRRR (SEQ ID NO: 43)
X-GRKARRQRRR (SEQ ID NO: 44)
X-RKARRQRRR (SEQ ID NO: 45)
X-GRKKARQRRR (SEQ ID NO: 46)
X-RKKARQRRR (SEQ ID NO: 47)
X-GRKKRRQARR (SEQ ID NO: 48)
X-RKKRRQARR (SEQ ID NO: 49)
X-GRKKRRQRAR (SEQ ID NO: 50)
X-RKKRRQRAR (SEQ ID NO: 51)
X-RRPRRPRRPRR (SEQ ID NO: 52)
X-RRARRARRARR (SEQ ID NO: 53)
X-RRRARRRARR (SEQ ID NO: 54)
X-RRRPRRRPRR (SEQ ID NO: 55)
X-RRPRRPRR (SEQ ID NO: 56)
X-RRARRARR (SEQ ID NO: 57)

X can represent a free amino terminus, one or more amino acids, or a conjugated moiety.

Active agents of the invention typically include an inhibitor peptide and an internalization peptide configured such that inhibitor peptide has a free C-terminus and an N-terminus linked to the C-terminus of the internalization peptide. In such agents, at least the four C-terminal residues of the inhibitor peptide and preferably the five C-terminal residues of the inhibitor peptide are L amino acids, and at least one of the remaining residues in the inhibitor peptide and internalization peptide is a D residue. Positions for inclusion of D residues can be selected such that D residues appear immediately after (i.e., on the C-terminal side) of any basic residue (i.e., arginine or lysine). Plasmin acts by cleaving the peptide bond on the C-terminal side of such basic residues. Inclusion of D residues flanking sites of cleavage, particularly on the C-terminal side of basic residues reduces or eliminates peptide cleavage. Any or all of residues on the C-terminal side of basic residues can be D residues. Any basic residues can also be D amino acids.

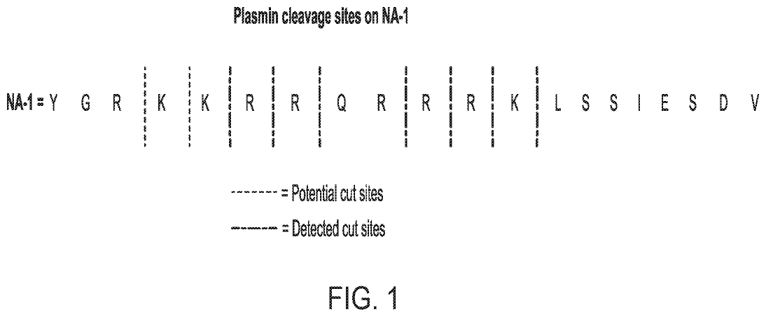

As an example, FIG. 1 shows a map of actual and potential plasmin cleavage sites in Tat-NR2B9c. There are seven actual sites (where cleavage has been detected) and two further potential sites, at which plasmin cleavage could occur. Some active agents include at least one D-amino acid in both the internalization peptide and inhibitor peptide. Some active agents include inhibitor peptides including D-amino acids at each position of the internalization peptide. Some active agents include D-amino acids at each position of the inhibitor peptide except the four or five C-terminal residues, which are L-amino acids. Some active agents include D-amino acids at each position of the internalization peptide, and each position of the inhibitor peptide except the last four or five C-terminal amino acid residues, which are L-amino acids.

Tat-NR2B9c, also known as NA-1 or nerinetide, has the amino acid sequence YGRKKRRQRRRKLSSIESDV (SEQ ID NO:58). Preferred active agents of the invention are variants of this sequence in which ESDV (SEQ ID NO:14) or IESDV (SEQ ID NO:5) are L-amino acids and at least one of the remaining amino acids is a D-amino acid. In some active agents at least the L or K residue at the eighth and ninth position from the C-terminus, or both, is or are D residues. In some active agents, at least one of the R, R, Q, R, R residues occupying the 6^{th}, 7^{th}, 8^{th}, 10^{th}, and 11^{th} positions from the N-terminus is a D residue. In some active agents all of these residues are D-residues. In some active agents, each of residues 4-8 and 10-13 residues are D-amino acids. In some active agents, each of residues 4-13 or 3-13 are D-amino acids. In some active agents, each of the eleven residues of the internalization peptide is a D-amino acid. Some exemplary active agents include ygrkkrrqrrrklsslESDV (SEQ ID NO:6) (also called NA-3), ygrkkrrqrrrklssiESDV (SEQ ID NO:59), ygrkkrrqrrrklsSIESDV (SEQ ID NO:60), ygrkkrrqrrrklSSIESDV (SEQ ID NO:61), ygrkkrrqrrrksslESDV (SEQ ID NO:7), ygrkkrrqrrrkslESDV (SEQ ID NO:8), or ygrkkrrqrrrklESDV (SEQ ID NO:9). Other active agents include variants of the above sequences in which the S at the third position from the C-terminal is replaced with T: ygrkkrrqrrrklssIETDV (SEQ ID NO:62), ygrkkrrqrrrklssiETDV (SEQ ID NO:63), ygrkkrrqrrrklsSIETDV (SEQ ID NO:64), ygrkkrrqrrrkssIETDV (SEQ ID NO:65), ygrkkrrqrrrksIETDV (SEQ ID NO:66), and ygrkkrrqrrrkIETDV (SEQ ID NO:67), Active agents include ygrkkrrqrrrlESDV (SEQ ID NO:68), (D-Tat-L-2B5c) and ygrkkrrqrrrIETDV (SEQ ID NO:69).

The invention also includes an active agent comprising an internalization peptide linked, e.g., as a fusion peptide, to an inhibitor peptide, which inhibits PSD-95 binding to NOS and/or NMDAR2B, wherein the internalization peptide has an amino acid sequence comprising YGRKKRRQRRR (SEQ ID NO:1), GRKKRRQRRR (SEQ ID NO:11), or RKKRRQRRR (SEQ ID NO:13) and the inhibitor peptide has a sequence comprising KLSSIESDV (SEQ ID NO:2), or a variant thereof with up to 1, 2, 3, 4, or 5 substitutions or deletions total in the internalization peptide and inhibitor peptide. In such active agents at least the four or five C-terminal amino acids of the inhibitor peptide are L-amino acids, and a contiguous segment of amino acids including all of the R and K residues and the residue immediately C-terminal to the most C-terminal R or K residue are D-amino acids. Thus, in a peptide having the sequence YGRKKRRQRRRKLSSIESDV (SEQ ID NO:58), a contiguous segment from the first R to the L residue are D-amino acids.

One example of permitted substitutions is provided by the motif [E/D/N/Q]-[S/T]-[D/E/Q/N]-[V/L] (SEQ ID NO:3) at the C-terminus of the inhibitor peptide. For example, the third amino acid from the C-terminus can be S or T. Preferably each of the five C-terminal amino acids of the inhibitor peptide are L-amino acids. Optionally every other amino acid is a D-amino acid as in the active agent ygrkkrrqrrrklsslESDV, wherein the lower case letter are D-amino acids and the upper case letters are L-amino acids.

Preferred active agents have enhanced stability (e.g., by half-life) in rat or human plasma compared with Tat-NR2B9c or an otherwise identical all L-active agent. Stability can be measured as in the examples. Preferred active agents have enhanced plasmin resistance compared with Tat-NR2B9c or an otherwise identical all L active agent. Plasmin resistance can be measured as in the examples. Active agents preferably bind to PSD-95 within 1.5-fold, 2- fold, 3 fold or 5- fold of Tat-NR2B9c (all L) or an otherwise identical all L peptide or have indistinguishable binding within experimental error. Preferred active agents compete for binding with Tat-NR2B9c or a peptide containing the last 15-20 amino acids of a NMDA Receptor subunit 2 sequence that contains the PDZ binding domain for binding to PSD-95 (e.g., a ten-fold excess of active agent reduces Tat-NR2B9c binding) by at least 10%, 25% or 50%. Competition provides an indication that the active agent binds to the same or overlapping binding site as Tat-NR2B9c. Possession of the same or overlapping binding sites can also be shown by alanine mutagenesis of PSD-95. If mutagenesis of the same or overlapping set of residues reduces binding of an active agent and Tat-NR2B9c, then the active agent and TAT-NR2B9c bind to the same or overlapping sites on PSD-95.

Active agents of the invention can contain modified amino acid residues for example, residues that are N-alkylated. N-terminal alkyl modifications can include e.g., N-Methyl, N-Ethyl, N-Propyl, N-Butyl, N-Cyclohexylmethyl, N-Cyclyhexylethyl, N-Benzyl, N-Phenylethyl, N-phenylpropyl, N-(3,4-Dichlorophenyl)propyl, N-(3,4-Difluorophenyl)propyl, and N-(Naphthalene-2-yl)ethyl). Active agents can also include retro peptides. A retro peptide has a reverse amino acid sequence. Peptidomimetics also include retro inverso peptides in which the order of amino acids is reversed from so the originally C-terminal amino acid appears at the N-terminus and D-amino acids are used in place of L-amino (e.g., acids vdseisslkrrrqrrkkrgy, also known as RI-NA-1).

Appropriate pharmacological activity of peptides, peptidomimetics or other agent can be confirmed if desired, using previously described rat models of stroke before testing in the primate and clinical trials described in the present application. Peptides or peptidomimetics can also be screened for capacity to inhibit interactions between PSD-95 and NMDAR 2B using assays described in e.g., US 20050059597, which is incorporated by reference. Useful peptides typically have IC50 values of less than 50 μM, 25 μM, 10 μM, 0.1 μM or 0.01 μM in such an assay. Preferred peptides typically have an IC50 value of between 0.001-1 μM, and more preferably 0.001-0.05, 0.05-0.5 or 0.05 to 0.1 μM. When a peptide or other agent is characterized as inhibiting binding of one interaction, e.g., PSD-95 interaction to NMDAR2B, such description does not exclude that the peptide or agent also inhibits another interaction, for example, inhibition of PSD-95 binding to nNOS.

Peptides such as those just described can optionally be derivatized (e.g., acetylated, phosphorylated, myristoylated, geranylated, pegylated and/or glycosylated) to improve the binding affinity of the inhibitor, to improve the ability of the inhibitor to be transported across a cell membrane or to improve stability. As a specific example, for inhibitors in which the third residue from the C-terminus is S or T, this residue can be phosphorylated before use of the peptide.

Internalization peptides can be attached to inhibitor peptides by conventional methods. For example, the inhibitor peptides can be joined to internalization peptides by chemical linkage, for instance via a coupling or conjugating agent. Numerous such agents are commercially available and are reviewed by S. S. Wong, Chemistry of Protein Conjugation and Cross-Linking, CRC Press (1991). Some examples of cross-linking reagents include J-succinimidyl 3-(2-pyridyldithio) propionate (SPDP) or N,N′-(1,3-phenylene) bismaleimide; N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents include p,p′-difluoro-m, m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

A linker, e.g., a polyethylene glycol linker, can be used to dimerize the active moiety of the peptide or the peptidomimetic to enhance its affinity and selectivity towards proteins containing tandem PDZ domains. See e.g., Bach et al., (2009) Angew. Chem. Int. Ed. 48:9685-9689 and WO 2010/004003. A PL motif-containing peptide is preferably dimerized via joining the N-termini of two such molecules, leaving the C-termini free. Bach further reports that a pentamer peptide IESDV (SEQ ID NO:5) from the C-terminus of NMDAR 2B was effective in inhibiting binding of NMDAR 2B to PSD-95. IETDV (SEQ ID NO:22) can also be used instead of IESDV (SEQ ID NO:5). Optionally, about 2-10 copies of a PEG can be joined in tandem as a linker. Optionally, the linker can also be attached to an internalization peptide or lipidated to enhance cellular uptake. Examples of illustrative dimeric inhibitors are shown below (see Bach et al., PNAS 109 (2012) 3317-3322). Any of the PSD-95 inhibitors disclosed herein can be used instead of IETDV, and any internalization peptide or lipidating moiety can be used instead of tat. Other linkers to that shown can also be used.

Internalization peptides can also be linked to inhibitor peptide as fusion peptides, preferably with the C-terminus of the internalization peptide linked to the N-terminus of the inhibitor peptide leaving the inhibitor peptide with a free C-terminus.

Instead of or as well as linking a peptide to an internalization peptide, such a peptide can be linked to a lipid (lipidation) to increase hydrophobicity of the conjugate relative to the peptide alone and thereby facilitate passage of the linked peptide across cell membranes and/or across the brain barrier. Lipidation is preferably performed on the N-terminal amino acid but can also be performed on internal amino acids, provided the ability of the peptide to inhibit interaction between PSD-95 and NMDAR 2B is not reduced by more than 50%. Preferably, lipidation is performed on an amino acid other than one of the five most C-terminal amino acids. Lipids are organic molecules more soluble in ether than water and include fatty acids, glycerides and sterols. Suitable forms of lipidation include myristoylation, palmitoylation or attachment of other fatty acids preferably with a chain length of 10-20 carbons, such as lauric acid and stearic acid, as well as geranylation, geranylgeranylation, and isoprenylation. Lipidations of a type occurring in posttranslational modification of natural proteins are preferred. Lipidation with a fatty acid via formation of an amide bond to the alpha-amino group of the N-terminal amino acid of the peptide is also preferred. Lipidation can be by peptide synthesis including a prelipidated amino acid, be performed enzymatically in vitro or by recombinant expression, by chemical crosslinking or chemical derivatization of the peptide. Amino acids modified by myristoylation and other lipid modifications are commercially available. Use of a lipid instead of an internalization peptide reduces the number of K and R residues providing sites of plasmin cleavage. Some exemplary lipidated molecules include KLSSIESDV (SEQ ID NO:2), kISSIESDV (SEQ ID NO:70), ISSIESDV (SEQ ID NO:71), LSSIESDV (SEQ ID NO:72), SSIESDV (SEQ ID NO:73), SIESDV (SEQ ID NO:74), IESDV (SEQ ID NO:5), KLSSIETDV (SEQ ID NO:12), kISSIETDV (SEQ ID NO:75), ISSIETDV (SEQ ID NO:76), LSSIETDV (SEQ ID NO:77), SSIETDV (SEQ ID NO:78), SIETDV (SEQ ID NO:79), IETDV (SEQ ID NO:22) with lipidation preferably at the N-terminus.

Inhibitor peptides, optionally fused to internalization peptides, can be synthesized by solid phase synthesis or recombinant methods. Peptidomimetics can be synthesized using a variety of procedures and methodologies described in the scientific and patent literature, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

III. Salts

Peptides of the type described above are typically made by solid state synthesis. Because solid state synthesis uses trifluoroacetate (TFA) to remove protecting groups or remove peptides from a resin, peptides are typically initially produced as trifloroacetate salts. The trifluoroacetate can be replaced with another anion by for example, binding the peptide to a solid support, such as a column, washing the column to remove the existing counterion, equilibrating the column with a solution containing the new counterion and then eluting the peptide, e.g., by introducing a hydrophobic solvent such as acetonitrile into the column. Replacement of trifluoroacetate with acetate can be done with an acetate wash as the last step before peptide is eluted in an otherwise conventional solid state synthesis. Replacing trifluoroacetate or acetate with chloride can be done with a wash with ammonium chloride followed by elution. Use of a hydrophobic support is preferred and preparative reverse phase HPLC is particularly preferred for the ion exchange. Trifluoroacetate can be replaced with chloride directly or can first be replaced by acetate and then the acetate replaced by chloride.

Counterions, whether trifluoroacetate, acetate or chloride, bind to positively charged atoms on Tat-NR2B9c and D-variants thereof, particularly the N-terminal amino group and amino side chains arginine and lysine residues. Although practice of the invention, it is not dependent on understanding the exact stoichiometry of peptide to anion in a salt of Tat-NR2B9c and its D-variants, it is believed that up to about 9 counterion molecules are present per molecule of salt.

Although replacement of one counterion by another takes place efficiently, the purity of the final counterion may be less than 100%. Thus, reference to a chloride salt of Tat-NR2B9c or its D-variants described herein means that in a preparation of the salt, chloride is the predominant anion by weight (or moles) over all other anions present in the aggregate in the salt. In other words, chloride constitutes greater than 50% and preferably greater than 75%, 95%, 99%, 99.5% or 99.9% by weight or moles of the all anions present in the salt. In such a salt or formulation prepared from the salt, acetate and trifluoroacetate in combination and individually constitutes less than 50%, 25%, 5%, 0.5% or 0.1 of the anions in the salt or formulation by weight or moles.

IV. Formulations

Active agents can be incorporated into liquid formulation or lyophilized formulations. A liquid formulation can include a buffer, salt and water. A preferred buffer is sodium phosphate. A preferred salt is sodium chloride. The pH can be e.g., pH7.0 or about physiological.

Lyophilized formulations can be prepared from a prelyophilized formulation comprising an active agent, a buffer, a bulking agent and water. Other components, such as cryo or lyopreservatives, a tonicity agent pharmaceutically acceptable carriers and the like may or may be present. A preferred active agent is a chloride salt of ygrkkrrqrrrklsslESDV (SEQ ID NO:6). A preferred buffer is histidine. A preferred bulking agent is trehalose. Trehalose also serves as a cryo and lyo-preservative. An exemplary prelyophilized formulation comprises the active agent, histidine (10-100 mM, 15-100 mM 15-80 mM, 40-60 mM or 15-60 mM, for example, 20 mM or optionally 50 mM, or 20-50 mM)) and trehalose (50-200 mM, preferably 80-160 mM, 100-140 mM, more preferably 120 mM). The pH is 5.5 to 7.5, more preferably, 6-7, more preferably 6.5. The concentration of active agent is 20-200 mg/ml, preferably 50-150 mg/ml, more preferably 70-120 mg/ml or 90 mg/ml. Thus, an exemplary prelyophilized formulation is 20 mM histidine, 120 mM trehalose, and 90 mg/ml chloride salt of active agent. Optionally an acetylation scavenger, such as lysine can be included, as described in U.S. Pat. No. 10,206,878, to further reduce any residual acetate or trifluoroacetate in the formulation

After lyophilization, lyophilized formulations have a low-water content, preferably from about 0%-5% water, more preferably below 2.5% water by weight. Lyophilized formulations can be stored in a freezer (e.g., −20 or −70° C.), in a refrigerator (0-40° C.) or at room temperature (20-25° C.).

Active agents can be reconstituted in an aqueous solution, preferably water for injection or optionally normal saline (0.8-1.0% saline and preferably 0.9% saline). Reconstitution can be to the same or a smaller or larger volume than the prelyophilized formulation. Preferably, the volume is larger post-reconstitution than before (e.g., 3-6 times larger). For example, a prelyophilization volume of 3-5 ml can be reconstituted as a volume of 10 mL, 12 mL, 13.5 ml, 15 mL or 20 mL or 10-20 mL among others. After reconstitution, the concentration of histidine is preferably 2-20 mM, e.g., 2-7 mM, 4.0-6.5 mM, 4.5 mM or 6 mM; the concentration of trehalose is preferably 15-45 mM or 20-40 mM or 25-27 mM or 35-37 mM. The concentration of lysine is preferably 100-300 mM, e.g., 150-250 mM, 150-170 mM or 210-220 mM. The active agent is preferably at a concentration of 10-30 mg/ml, for example 15-30, 18-20, 20 mg/ml of active agent or 25-30, 26-28 or 27 mg/mL active agent. An exemplary formulation after reconstitution has 4-5 mM histidine, 26-27 mM trehalose, 150-170 mM lysine and 20 mg/ml active agent (with concentrations rounded to the nearest integer). A second exemplary formulation after reconstitution has 5-7 mM histidine, 35-37 mM trehalose, 210-220 mM lysine and 26-28 mg/ml active agent (with concentrations rounded to the nearest integer). The reconstituted formulation can be further diluted before administration such as by adding into a fluid bag containing normal saline.

IV. Diseases

The active agents are useful in treating a variety of diseases, particularly neurological diseases, and especially diseases mediated in part by excitotoxity. Such diseases and conditions include stroke, epilepsy, hypoxia, subarachnoid hemorrhage, traumatic injury to the CNS not associated with stroke such as traumatic brain injury and spinal cord injury, other cerebral ischemia, Alzheimer's disease and Parkinson's disease. Such conditions can also include disorders or diseases of the eye or ear, including retinopathies, retinal ischemia associated other ocular disorders, or tinnitus. Other neurological diseases treatable by active agents of the invention not known to be associated with excitotoxicity include anxiety and pain (either neuropathic or inflammatory).

A stroke is a condition resulting from impaired blood flow in the CNS regardless of cause. Potential causes include embolism, hemorrhage and thrombosis. Some neuronal cells die immediately as a result of impaired blood flow. These cells release their component molecules including glutamate, which in turn activates NMDA receptors, which raise intracellular calcium levels, and intracellular enzyme levels leading to further neuronal cell death (the excitotoxicity cascade). The death of CNS tissue is referred to as infarction. Infarction Volume (i.e., the volume of dead neuronal cells resulting from stroke in the brain) can be used as an indicator of the extent of pathological damage resulting from stroke. The symptomatic effect depends both on the volume of an infarction and where in the brain it is located. Disability index can be used as a measure of symptomatic damage, such as the Rankin Stroke Outcome Scale (Rankin, Scott Med J; 2:200-15 (1957)) and the Barthel Index. The Rankin Scale is based on assessing directly the global conditions of a patient as follows.
0: No symptoms at all
1: No significant disability despite symptoms; able to carry out all usual duties and activities.
2: Slight disability; unable to carry out all previous activities but able to look after own affairs without assistance.
3: Moderate disability requiring some help, but able to walk without assistance
4: Moderate to severe disability; unable to walk without assistance and unable to attend to own bodily needs without assistance.
5: Severe disability; bedridden, incontinent, and requiring constant nursing care and attention.

The Barthel Index is based on a series of questions about the patient's ability to carry out 10 basic activities of daily living resulting in a score between 0 and 100, a lower score indicating more disability (Mahoney et al, Maryland State Medical Journal 14:56-61 (1965)).

Alternatively stroke severity/outcomes can be measured using the NIH stroke scale, available at world wide web ninds.nih.gov/doctors/NIH Stroke ScaleJBooklet.pdf.

The scale is based on the ability of a patient to carry out 11 groups of functions that include assessments of the patient's level of consciousness, motor, sensory and language functions.

An ischemic stroke refers more specifically to a type of stroke that caused by blockage of blood flow to the brain. The underlying condition for this type of blockage is most commonly the development of fatty deposits lining the vessel walls. This condition is called atherosclerosis. These fatty deposits can cause two types of obstruction. Cerebral thrombosis refers to a thrombus (blood clot) that develops at the clogged part of the vessel “Cerebral embolism” refers generally to a blood clot that forms at another location in the circulatory system, usually the heart and large arteries of the upper chest and neck. A portion of the blood clot then breaks loose, enters the bloodstream and travels through the brain's blood vessels until it reaches vessels too small to let it pass. A second important cause of embolism is an irregular heartbeat, known as arterial fibrillation. It creates conditions in which clots can form in the heart, dislodge and travel to the brain. Additional potential causes of ischemic stroke are hemorrhage, thrombosis, dissection of an artery or vein, a cardiac arrest, shock of any cause including hemorrhage, and iatrogenic causes such as direct surgical injury to brain blood vessels or vessels leading to the brain or cardiac surgery. Ischemic stroke accounts for about 83 percent of all cases of stroke.

Transient ischemic attacks (TIAs) are minor or warning strokes. In a TIA, conditions indicative of an ischemic stroke are present and the typical stroke warning signs develop. However, the obstruction (blood clot) occurs for a short time and tends to resolve itself through normal mechanisms. Patients undergoing heart surgery are at particular risk of transient cerebral ischemic attack.

Hemorrhagic stroke accounts for about 17 percent of stroke cases. It results from a weakened vessel that ruptures and bleeds into the surrounding brain. The blood accumulates and compresses the surrounding brain tissue. The two general types of hemorrhagic strokes are intracerebral hemorrhage and subarachnoid hemorrhage. Hemorrhagic stroke result from rupture of a weakened blood vessel ruptures. Potential causes of rupture from a weakened blood vessel include a hypertensive hemorrhage, in which high blood pressure causes a rupture of a blood vessel, or another underlying cause of weakened blood vessels such as a ruptured brain vascular malformation including a brain aneurysm, arteriovenous malformation (AVM) or cavernous malformation. Hemorrhagic strokes can also arise from a hemorrhagic transformation of an ischemic stroke which weakens the blood vessels in the infarct, or a hemorrhage from primary or metastatic tumors in the CNS which contain abnormally weak blood vessels. Hemorrhagic stroke can also arise from iatrogenic causes such as direct surgical injury to a brain blood vessel. An aneurysm is a ballooning of a weakened region of a blood vessel. If left untreated, the aneurysm continues to weaken until it ruptures and bleeds into the brain. An arteriovenous malformation (AVM) is a cluster of abnormally formed blood vessels. A cavernous malformation is a venous abnormality that can cause a hemorrhage from weakened venous structures. Any one of these vessels can rupture, also causing bleeding into the brain. Hemorrhagic stroke can also result from physical trauma. Hemorrhagic stroke in one part of the brain can lead to ischemic stroke in another through shortage of blood lost in the hemorrhagic stroke.

One patient class amenable to treatments are patients undergoing a surgical procedure that involves or may involve a blood vessel supplying the brain, or otherwise on the brain or CNS. Some examples are patients undergoing cardiopulmonary bypass, carotid stenting, diagnostic angiography of the brain or coronary arteries of the aortic arch, vascular surgical procedures and neurosurgical procedures. Additional examples of such patients are discussed in section IV above. Patients with a brain aneurysm are particularly suitable. Such patients can be treated by a variety of surgical procedures including clipping the aneurysm to shut off blood, or performing endovascular surgery to block the aneurysm with small coils or introduce a stent into a blood vessel from which an aneurysm emerges, or inserting a microcatheter. Endovascular procedures are less invasive than clipping an aneurysm and are associated with a better patient outcome but the outcome still includes a high incidence of small infarctions. Such patients can be treated with an inhibitor of PSD95 interaction with NMDAR 2B and particularly the agents described above. The timing of administration relative to performing surgery can be as described above for the clinical trial.

Another class of patients amenable to treatment are patients having a subarachnoid hemorrhage with or without an aneurysm (see U.S. 61/570,264). Another class of patients is those with ischemic strokes who are candidates for endovascular thrombectomy to remove the clot, such as the ESCAPE-NA1 trial (NCT02930018). Drug can be administered before or after the surgery to remove the clot, and is expected to improve outcome from both the stroke itself and any potential iatrogenic strokes associated with the procedures as discussed supra. Another example is those who have been diagnosed with a potential stroke without the use of imaging criteria and receive treatment within hours of the stroke, preferably within the first 3 hours but optionally the first 6, 9 or 12 hour after stroke onset (similar to NCT02315443).

IV. Effective Regimes of Administration

An active agent is administered in an amount, frequency and route of administration effective to cure, reduce or inhibit further deterioration of at least one sign or symptom of a disease in a patient having the disease being treated. A therapeutically effective amount (before administration) or therapeutically effective plasma concentration after administration means an amount or level of active agent sufficient significantly to cure, reduce or inhibit further deterioration of at least one sign or symptom of the disease or condition to be treated in a population of patients (or animal models) suffering from the disease treated with an agent of the invention relative to the damage in a control population of patients (or animal models) suffering from that disease or condition who are not treated with the agent. The amount or level is also considered therapeutically effective if an individual treated patient achieves an outcome more favorable than the mean outcome in a control population of comparable patients not treated by methods of the invention. A therapeutically effective regime involves the administration of a therapeutically effective dose at a frequency and route of administration needed to achieve the intended purpose.

For a patient suffering from stroke or other ischemic condition, the active agent is administered in a regime comprising an amount frequency and route of administration effective to reduce the damaging effects of stroke or other ischemic condition. When the condition requiring treatment is stroke, the outcome can be determined by infarction volume or disability index, and a dosage is considered therapeutically effective if an individual treated patient shows a disability of two or less on the Rankin scale and 75 or more on the Barthel scale, or if a population of treated patients shows a significantly improved (i.e., less disability) distribution of scores on a disability scale than a comparable untreated population, see Lees et at L, N Engl J Med 2006; 354:588-600. A single dose of agent can be sufficient for treatment of stroke.

The invention also provides methods and formulations for the prophylaxis of a disorder in a subject at risk of that disorder. Usually such a subject has an increased likelihood of developing the disorder (e.g., a condition, illness, disorder or disease) relative to a control population. The control population for instance can comprise one or more individuals selected at random from the general population (e.g., matched by age, gender, race and/or ethnicity) who have not been diagnosed or have a family history of the disorder. A subject can be considered at risk for a disorder if a “risk factor” associated with that disorder is found to be associated with that subject. A risk factor can include any activity, trait, event or property associated with a given disorder, for example, through statistical or epidemiological studies on a population of subjects. A subject can thus be classified as being at risk for a disorder even if studies identifying the underlying risk factors did not include the subject specifically. For example, a subject undergoing heart surgery is at risk of transient cerebral ischemic attack because the frequency of transient cerebral ischemic attack is increased in a population of subjects who have undergone heart surgery as compared to a population of subjects who have not.

Other common risk factors for stroke include age, family history, gender, prior incidence of stroke, transient ischemic attack or heart attack, high blood pressure, smoking, diabetes, carotid or other artery disease, atrial fibrillation, other heart diseases such as heart disease, heart failure, dilated cardiomyopathy, heart valve disease and/or congenital heart defects; high blood cholesterol, and diets high in saturated fat, trans fat or cholesterol.

In prophylaxis, an active agent is administered to a patient at risk of a disease but not yet having the disease in an amount, frequency and route sufficient to prevent, delay or inhibit development of at least one sign or symptom of the disease. A prophylactically effective amount before administration or plasma level after administration means an amount or level of agent sufficient significantly to prevent, inhibit or delay at least one sign or symptom of the disease in a population of patients (or animal models) at risk of the disease relative treated with the agent compared to a control population of patients (or animal models) at risk of the disease not treated with an active agent of the invention. The amount or level is also considered prophylactically effective if an individual treated patient achieves an outcome more favorable than the mean outcome in a control population of comparable patients not treated by methods of the invention. A prophylactically effective regime involves the administration of a prophylactically effective dose at a frequency and route of administration needed to achieve the intended purpose. For prophylaxis of stroke in a patient at imminent risk of stroke (e.g., a patient undergoing heart surgery), a single dose of agent is usually sufficient.

Depending on the agent, administration can be parenteral, intravenous, intrapulmonary, nasal, oral, subcutaneous, intra-arterial, intracranial, intrathecal, intraperitoneal, topical, intranasal or intramuscular.

Tat-NR2B9c has previously been administered to humans by single dose intravenous infusion at 2.6 mg/kg. The present active agents can achieved greater CMax and AUC than Tat-NR2B9c when administered by non-intravenous routes, such as subcutaneous, intranasal or intramuscular, because their longer half-life compensates for the additional time required for the active agents to reach the plasma. Administration by such non-intravenous routes also allows higher dosages to be administered without releasing significant amounts of histamine due to mast cell degranulation. For example, doses of up to about 10 mg/kg can be used without releasing significant histamine, and even doses up to 25 mg/kg release detectable histamine but much less than administration of the same dose intravenously.

Thus, depending on the route of administration and whether an anti-inflammatory is co-administered to reduce histamine release or its downstream effects, a range of dosages can be administered. For intravenous administration, the claimed agents can be administered at similar dosage as Tat-NR2B9c without anti-inflammatory e.g., up to 3 mg/kg, 0.1-3 mg/kg, 2-3 mg/kg or 2.6 mg/kg, or at higher dosages, e.g., at least 5, 10, 15, 20 or 25 mg/kg with an anti-inflammatory. For routes such as subcutaneous, intranasal, intrapulmonary or intramuscular, the dose can be up to 10, 15, or 20 mg/kg without an anti-inflammatory or more than 10, 15, 20, 25 or 50 mg/kg with an anti-inflammatory. The need for an-inflammatory at higher doses can alternatively be reduced or eliminated by administration of the active agent over a longer time period (e.g., administration in less than 1 minute, 1-10 minutes, and greater than ten minutes constitute alternative regimes in which for constant dosage histamine release and need for an anti-inflammatory is reduced or eliminated with increased time period).

The active agents can be administered as a single dose or as a multi-dose regime. A single dose regime can be used for treatment of an acute condition, such as acute ischemic stroke, to reduce infarction and cognitive deficits. Such a dose can be administered before onset of the condition if the timing of the condition is predictable such as with a subject undergoing neurovascular surgery, or within a window after the condition has developed (e.g., up to 1, 3, 6 or 12 hours later).

A multi-dose regime can be designed to maintain the active agent at a detectable level in the plasma over a prolonged period of time, such as at least 1, 3, 5 or 10 days, or at least a month, three months, six months or indefinitely. For example, the active agents can be administered every hour, 2, 3, 4, 6, or 12 times per day, daily, every other day, weekly and so forth. Such a regime can reduce initial deficits from an acute condition as for single dose administration and thereafter promote recovery from such deficits as still develop. Such a regime can also be used for treating chronic conditions, such as Alzheimer's and Parkinson's disease. Active agents are sometimes incorporated into a controlled release formulation for use in a multi-dose regime.

Active agents can be prepared with carriers that protect the compound against rapid elimination from the body, such as controlled formulations or coatings. Such carriers (also known as modified, delayed, extended or sustained release or gastric retention dosage forms, such as the Depomed GR™ system in which agents are encapsulated by polymers that swell in the stomach and are retained for about eight hours, sufficient for daily dosing of many drugs). Controlled release systems include microencapsulated delivery systems, implants and biodegradable, biocompatible polymers such as collagen, ethylene vinyl acetate, polyanhydrides, polyglycolic acid, polyorthoesters, polylactic acid, matrix controlled release devices, osmotic controlled release devices, multiparticulate controlled release devices, ion-exchange resins, enteric coatings, multilayered coatings, microspheres, nanoparticles, liposomes, and combinations thereof. The release rate of an active agent can also be modified by varying the particle size of the active agent: Examples of modified release include, e.g., those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

V. Co-Administration with Anti-Inflammatories

Depending on the dose and route of administration the active agents of the invention can induce an inflammatory response characterized by mast cell degranulation and release of histamine and its sequelae. For example, dosages of at least 3 mg/kg are associated with histamine release for IV administration, and at least 10 mg/kg for other routes.

A wide variety of anti-inflammatory agents are readily available to inhibit one or more aspects of the of the inflammatory response. A preferred class of anti-inflammatory agent is mast cell degranulation inhibitors. This class of compounds includes cromolyn (5,5′-(2-hydroxypropane-1,3-diyl)bis(oxy)bis(4-oxo-4H-chromene-2-carboxylic acid) (also known as cromoglycate), and 2-carboxylatochromon-5′-yl-2-hydroxypropane derivatives such as bis(acetoxymethyl), disodium cromoglycate, nedocromil (9-ethyl-4,6-dioxo-10-propyl-6,9-dihydro-4H-pyrano[3,2-g]quinoline-2,8-di-carboxylic acid) and tranilast (2-{[(2E)-3-(3,4-dimethoxyphenyl)prop-2-enoyl]amino}), and lodoxamide (2-[2-chloro-5-cyano-3-(oxaloamino)anilino]-2-oxoacetic acid). Reference to a specific compound includes pharmaceutically acceptable salts of the compound Cromolyn is readily available in formulations for nasal, oral, inhaled or intravenous administration. Although practice of the invention is not dependent on an understanding of mechanism, it is believed that these agents act at an early stage of inflammatory response induced by an internalization peptide and are thus most effective at inhibiting development of its sequelae including a transient reduction in blood pressure. Other classes of anti-inflammatory agent discussed below serve to inhibit one or more downstream events resulting from mast cell degranulation, such as inhibiting histamine from binding to an H1 or H2 receptor, but may not inhibit all sequelae of mast cell degranulation or may require higher dosages or use in combinations to do so. Table 2 below summarizes the names, chemical formulate and FDA status of several mast cell degranulation inhibitors that can be used with the invention.

TABLE 2

| | | | FDA |
|---|---|---|---|
| Drug Name | Alternative Names | Chemical Formula | status |
| Azelastine | Astelin, Optivar | 4-[(4-chlorophenyl)methyl]-2- | Approved |
| | | (1-methylazepan-4-yl)phthalazin-1- | |
| | | one | |
| Bepotastine | Bepotastine besilate, | 4-[4-[(4-chlorophenyl)-pyridin-2- | Approved |
| | Betotastine besilate, | ylmethoxy]piperidin-1- | |
| | TAU-284DS, bepotastine | yl]butanoic acid | |
| Chlorzoxazone | Biomioran, EZE-DS, Escoflex, | 5-chloro-3H-1,3-benzoxazol-2- | Approved |
| | Flexazone, Mioran, Miotran, | one | |
| | Myoflexin, Myoflexine, Neoflex, | | |
| | Paraflex, Parafon Forte Dsc, | | |
| | Pathorysin, Relaxazone, Remular, | | |
| | Remular-S, Solaxin, Strifon | | |
| | Forte Dsc, Usaf Ma-10 | | |
| Cromolyn | Cromoglycate, Chromoglicate, | 5-[3-(2-carboxy-4-oxochromen- | Approved |
| | Chromoglicic Acid, Aarane, | 6-yl)oxy-2-hydroxypropoxy]-4- | |
| | Alercom, Alerion, Allergocrom, | oxochromene-2-carboxylic acid | |
| | ApoCromolyn, Children't | | |
| | Nasalcrom, Colimune, Crolom, | | |
| | Cromolyn Nasal Solution, | | |
| | Cromoptic, Cromovet, Fivent, | | |
| | Gastrocrom, Gastrofrenal, | | |
| | GenCromoglycate, Inostral, | | |
| | Intal, Intal, Inhaler, Intal, | | |
| | Syncroner, Introl, Irtan, | | |
| | Lomudal, Lomupren, Lomusol, | | |
| | Lomuspray, Nalcrom, Nalcron, | | |
| | Nasalcrom, Nasmil, Opticrom, | | |
| | Opticron, Rynacrom, Sofro, | | |
| | Vistacrom, Vividrin | | |
| Epinastine | Elestat | C16H15N3, CAS 80012-43-7 | Approved |
| Isoproterenol | Aerolone, Aleudrin, Aleudrine, | 4-[1-hydroxy-2-(propan-2- | Approved |
| | Aludrin, Aludrine, Asiprenol, | ylamino)ethyl]benzene-1,2-diol | |
| | Asmalar, Assiprenol, Bellasthman, | | |
| | Bronkephrine, Euspiran, Isadrine, | | |
| | Isonorene, Isonorin, Isorenin, | | |
| | Isuprel, Isuprel Mistometer, | | |
| | Isupren, | | |
| | Medihaler-Iso, NeoEpinine, | | |
| | Neodrenal, Norisodrine, m | | |
| | Norisodrine, Aerotrol, | | |
| | Novodrin, Proternol, | | |
| | Respifral, Saventrine, | | |
| | Vapo-Iso | | |
| Ketotifen | Zaditor | C19H19NOS, CAS 34580-14-8 | Approved |
| Lodoxamide | Alomide | N,N′-(2-chloro-5-cyano-m- | Approved |
| (lodoxamide | | phenylene)dioxamic acid | |
| tromethamine) | | tromethamine salt | |
| Nedocromil | Alocril, Nedocromil | 9-ethyl-4,6-dioxo-10- | Approved |
| | [USAN:BAN:INN], | propylpyrano[5,6-g]quinoline- | |
| | Tilade | 2,8-dicarboxylic acid | |
| Olopatadine | Olopatadine Hydrochloride | 2-[(11Z)-11-(3- | Approved |
| | Patanol | dimethylaminopropylidene)-6H- | |
| | | benzo[c][2]benzoxepin-2- | |
| | | yl]acetic acid | |
| Pemirolast | Alamast | 9-methyl-3-(2H-tetrazol-5- | Approved |
| | | yl)pyrido[2,1-b]pyrimidin-4-one | |
| Pirbuterol | Maxair | 6-[2-(tert-butylamino)-1- | Approved |
| | | hydroxyethyl]-2- | |
| | | (hydroxymethyl)pyridin-3-ol | |

Another class of anti-inflammatory agent is anti-histamine compounds. Such agents inhibit the interaction of histamine with its receptors thereby inhibiting the resulting sequelae of inflammation noted above. Many anti-histamines are commercially available, some over the counter. Examples of anti-histamines are azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines or terfenadine. Anti-histamines vary in their capacity to block anti-histamine in the CNS as well as peripheral receptors, with second and third generation anti-histamines having selectivity for peripheral receptors. Acrivastine, Astemizole, Cetirizine, Loratadine, Mizolastine, Levocetirizine, Desloratadine, and Fexofenadine are examples of second and third generation anti-histamines. Anti-histamines are widely available in oral and topical formulations. Some other anti-histamines that can be used are summarized in Table 3 below.

TABLE 3

| | | | FDA |
|---|---|---|---|
| Drug Name | Alternative Names | Chemical Formula | status |
| Ketotifen | Ketotifen, Zaditor | C19H19NOS | Approved |
| fumarate | | | |
| Mequitazine | Butix, Instotal, Kitazemin, | 10-(1-azabicyclo[2.2.2]octan-8- | Approved |
| | Metaplexan, Mircol, Primalan, | ylmethyl)phenothiazine | |
| | Vigigan, Virginan, Zesulan | | |
| Dexbrompheniramine | Ilvan | (3S)-3-(4-bromophenyl)-N,N- | Approved |
| | | dimethyl-3-pyridin-2-ylpropan-1- | |
| | | amine | |
| Methdilazine | Bristaline, Dilosyn, Disyncram, | 10-[(1-methylpyrrolidin-3- | Approved |
| | Disyncran, Tacaryl, Tacaryl | yl)methyl]phenothiazine | |
| | hydrochloride, Tacazyl, Tacryl | | |
| Chlorpheniramine | Aller-Chlor, Allergican, Allergisan, | 3-(4-chlorophenyl)-N,N-dimethyl- | Approved |
| | Antagonate, Chlo-Amine, Chlor- | 3-pyridin-2-ylpropan-1-amine | |
| | Trimeton, Chlor-Trimeton Allergy, | | |
| | Chlor-Trimeton Repetabs, Chlor- | | |
| | Tripolon, Chlorate, Chloropiril, | | |
| | Cloropiril, Efidac 24 | | |
| | Chlorpheniramine Maleate, Gen- | | |
| | Allerate, Haynon, Histadur, | | |
| | Kloromin, Mylaramine, Novo- | | |
| | Pheniram, Pediacare Allergy | | |
| | Formula, Phenetron, Piriton, | | |
| | Polaramine, Polaronil, Pyridamal | | |
| | 100, Telachlor, Teldrin | | |
| Bromopheniramine | Bromfed, Bromfenex, Dimetane, | 3-(4-bromophenyl)-N,N-dimethyl- | Approved |
| | Veltane | 3-pyridin-2-ylpropan-1-amine | |
| Terbutaline | Brethaire, Brethine, Brican, Bricanyl, | 5-[2-(tert-butylamino)-1- | Approved |
| | Bricar, Bricaril, Bricyn | hydroxyethyl]benzene-1,3-diol | |
| pimecrolimus | Elidel | (3S,4R,5S,8R,9E,12S,14S,15R,16S, | Approved |
| | | 18R,19R,26aS)-3-{(E)-2-[(1R,3R,4S)- | as topical, |
| | | 4-Chloro-3-methoxycyclohexyl]-1- | Investigational as |
| | | methylvinyl}-8-ethyl- | oral |
| | | 5,6,8,11,12,13,14,15,16,17,18,19, | |
| | | 24,25,26,26a-hexadecahydro-5,19- | |
| | | dihydroxy-14,16-dimethoxy- | |
| | | 4,10,12,18-tetramethyl-15,19- | |
| | | epoxy-3H-pyrido[2,1- | |
| | | c][1,4]oxaazacyclotricosine- | |
| | | 1,7,20,21(4H,23H)-tetrone | |

Another class of anti-inflammatory agent useful in inhibiting the inflammatory response is corticosteroids. These compounds are transcriptional regulators and are powerful inhibitors of the inflammatory symptoms set in motion by release of histamine and other compounds resulting from mast cell degranulation. Examples of corticosteroids are Cortisone, Hydrocortisone (Cortef), Prednisone (Deltasone, Meticorten, Orasone), Prednisolone (Delta-Cortef, Pediapred, Prelone), Triamcinolone (Aristocort, Kenacort), Methyl prednisolone (Medrol), Dexamethasone (Decadron, Dexone, Hexadrol), and Betamethasone (Celestone). Corticosteriods are widely available in oral, intravenous and topical formulations.

Nonsteroidal anti-inflammatory drugs (NSAIDs) can also be used. Such drugs include aspirin compounds (acetylsalicylates), non-aspirin salicylates, diclofenac, diflunisal, etodolac, fenoprofen, flurbiprofen, ibuprofen, indomethacin, ketoprofen, meclofenamate, naproxen, naproxen sodium, phenylbutazone, sulindac, and tometin. However, the anti-inflammatory effects of such drugs are less effective than those of anti-histamines or corticosteroids. Stronger anti-inflammatory drugs such as azathioprine, cyclophosphamide, leukeran, and cyclosporine can also be used but are not preferred because they are slower acting and/or associated with side effects. Biologic anti-inflammatory agents, such as Tysabri® or Humira® can also be used but are not preferred for the same reasons.

Different classes of drugs can be used in combinations in inhibiting an inflammatory response. A preferred combination is a mast cell degranulation inhibitor and an anti-histamine.

In methods in which a pharmacological agent linked to an internalization peptide is administered with an anti-inflammatory agent, the two entities are administered sufficiently proximal in time that the anti-inflammatory agent can inhibit an inflammatory response inducible by the internalization peptide. The anti-inflammatory agent can be administered before, at the same time as or after the pharmacologic agent. The preferred time depends in part on the pharmacokinetics and pharmacodynamics of the anti-inflammatory agent. The anti-inflammatory agent can be administered at an interval before the pharmacologic agent such that the anti-inflammatory agent is near maximum serum concentration at the time the pharmacologic agent is administered. Typically, the anti-inflammatory agent is administered between 6 hours before the pharmacological agent and one hour after. For example, the anti-inflammatory agent can be administered between 1 hour before and 30 min after the pharmacological agent. Preferably the anti-inflammatory agent is administered between 30 minutes before and 15 minutes after the pharmacologic agent, and more preferably within 15 minutes before and the same time as the pharmacological agent. In some methods, the anti-inflammatory agent is administered before the pharmacological agent within a period of 15, 10 or 5 minutes before the pharmacological agent is administered. In some methods, the agent is administered 1-15, 1-10 or 1-5 minutes before the pharmacological agent.

When administration of an agent is not instantaneous, such as with intravenous infusion, the anti-inflammatory agent and pharmacological agent are considered to be administered at the same time if their periods of administration are co-extensive or overlap. Time periods of administration before administration start from the beginning of its administration. Time periods after administration start from the end of its administration. Time periods referring to the administration of the anti-inflammatory agent refer to the beginning of its administration.

When an anti-inflammatory agent is said to be able to inhibit the inflammatory response of a pharmacological agent linked to an internalization peptide what is meant is that the two are administered sufficiently proximate in time that the anti-inflammatory agent would inhibit an inflammatory response inducible by the pharmacological agent linked to the internalization peptide if such a response occurs in a particular patient, and does not necessarily imply that such a response occurs in that patient. Some patients are treated with a dose of pharmacological agent linked to an internalization peptide that is associated with an inflammatory response in a statistically significant number of patients in a controlled clinical or nonclinical trial. It can reasonably be assumed that a significant proportion of such patients although not necessarily all develop an anti-inflammatory response to the pharmacological agent linked to the internalization peptide. In some patients, signs or symptoms of an inflammatory response to the pharmacological agent linked to the internalization peptide are detected or detectable.

In clinical treatment of an individual patient, it is not usually possible to compare the inflammatory response from a pharmacological agent linked to an internalization peptide in the presence and absence of an anti-inflammatory agent. However, it can reasonably be concluded that the anti-inflammatory agent inhibits an anti-inflammatory response inducible by the peptide if significant inhibition is seen under the same or similar conditions of co-administration in a controlled clinical or pre-clinical trial. The results in the patient (e.g., blood pressure, heart rate, hives) can also be compared with the typical results of a control group in a clinical trial as an indicator of whether inhibition occurred in the individual patient. Usually, the anti-inflammatory agent is present at a detectable serum concentration at some point within the time period of one hour after administration of the pharmacologic agent. The pharmacokinetics of many anti-inflammatory agents is widely known and the relative timing of administration of the anti-inflammatory agent can be adjusted accordingly. The anti-inflammatory agent is usually administered peripherally, i.e., segregated by the blood brain barrier from the brain. For example, the anti-inflammatory agent can be administered orally, nasally, intravenously or topically depending on the agent in question. If the anti-inflammatory agent is administered at the same time as the pharmacologic agent, the two can be administered as a combined formulation or separately.

In some methods, the anti-inflammatory agent is one that does not cross the blood brain barrier when administered orally or intravenously at least in sufficient amounts to exert a detectable pharmacological activity in the brain. Such an agent can inhibit mast cell degranulation and its sequelae resulting from administration of the active agent in the periphery without itself exerting any detectable therapeutic effects in the brain. In some methods, the anti-inflammatory agent is administered without any co-treatment to increase permeability of the blood brain barrier or to derivatize or formulate the anti-inflammatory agent so as to increase its ability to cross the blood brain barrier. However, in other methods, the anti-inflammatory agent, by its nature, derivatization, formulation or route of administration, may by entering the brain or otherwise influencing inflammation in the brain, exert a dual effect in suppressing mast-cell degranulation and/or its sequelae in the periphery due to an internalization peptide and inhibiting inflammation in the brain. Strbian et al., WO 04/071531 have reported that a mast cell degranulation inhibitor, cromoglycate, administered i.c.v. but not intravenously has direct activity in inhibiting infarctions in an animal model.

In some methods, the patient is not also treated with the same anti-inflammatory agent co-administered with the active agent in the day, week or month preceding and/or following co-administration with the active agent. In some methods, if the patient is otherwise being treated with the same anti-inflammatory agent co-administered with the active agent in a recurring regime (e.g., same amount, route of delivery, frequency of dosing, timing of day of dosing), the co-administration of the anti-inflammatory agent with the active agent does not comport with the recurring regime in any or all of amount, route of delivery, frequency of dosing or time of day of dosing. In some methods, the patient is not known to be suffering from an inflammatory disease or condition requiring administration of the anti-inflammatory agent co-administered with the active agent in the present methods. In some methods, the patient is not suffering from asthma or allergic disease treatable with a mast cell degranulation inhibitor. In some methods, the anti-inflammatory agent and active agent are each administered once and only once within a window as defined above, per episode of disease, an episode being a relatively short period in which symptoms of disease are present flanked by longer periods in which symptoms are absent or reduced.

The anti-inflammatory agent is administered in a regime of an amount, frequency and route effective to inhibit an inflammatory response to an internalization peptide under conditions in which such an inflammatory response is known to occur in the absence of the anti-inflammatory. An inflammatory response is inhibited if there is any reduction in signs or symptoms of inflammation as a result of the anti-inflammatory agent. Symptoms of the inflammatory response can include redness, rash such as hives, heat, swelling, pain, tingling sensation, itchiness, nausea, rash, dry mouth, numbness, airway congestion. The inflammatory response can also be monitored by measuring signs such as blood pressure, or heart rate. Alternatively, the inflammatory response can be assessed by measuring plasma concentration of histamine or other compounds released by mast cell degranulation. The presence of elevated levels of histamine or other compounds released by mast cell degranulation, reduced blood pressure, skin rash such as hives, or reduced heart rate are indicators of mass cell degranulation. As a practical matter, the doses, regimes and routes of administration of most of the anti-inflammatory agents discussed above are available in the Physicians' Desk Reference and/or from the manufacturers, and such anti-inflammatories can be used in the present methods consistent with such general guidance.

VI. Co-Administration with Thrombolytic Agents

Plaques and blood clots (also known as emboli) causing ischemia can be dissolved, removed or bypassed by both pharmacological and physical means. The dissolving, removal of plaques and blood clots and consequent restoration of blood flow is referred to as reperfusion. One class of agents acts by thrombolysis. Thrombolytic agents work by promoting production of plasmin. Plasmin clears cross-linked fibrin mesh (the backbone of a clot), making the clot soluble and subject to further proteolysis by other enzymes, and restores blood flow in occluded blood vessels. Examples of thrombolytic agents include tissue plasminogen activator t-PA, alteplase (Activase), reteplase (Retavase), tenecteplase (TNKase), anistreplase (Eminase), streptokinase (Kabikinase, Streptase), and urokinase (Abbokinase).

Another class of drugs that can be used for reperfusion is vasodilators. These drugs act by relaxing and opening up blood vessels thus allowing blood to flow around an obstruction. Some examples of types of vasodilators alpha-adrenoceptor antagonists (alpha-blockers), Angiotensin receptor blockers (ARBs), Beta.sub.2-adrenoceptor agonists (.beta..sub.2-agonists), calcium-channel blockers (CCBs), centrally acting sympatholytics, direct acting vasodilators, endothelin receptor antagonists, ganglionic blockers, nitrodilators, phosphodiesterase inhibitors, potassium-channel openers, and renin inhibitors.

Another class of drugs that can be used for reperfusion is hypertensive drugs (i.e., drugs raising blood pressure), such as epinephrine, phenylephrine, pseudoephedrine, norepinephrine; norephedrine; terbutaline; salbutamol; and methylephedrine. Increased perfusion pressure can increase flow of blood around an obstruction.

Mechanical methods of reperfusion include angioplasty, catheterization, and artery bypass graft surgery, stenting, embolectomy, or endarterectomy. Such procedures restore plaque flow by mechanical removal of a plaque, holding a blood vessel open, so blood can flow around a plaque or bypassing a plaque.

Other mechanical methods of reperfusion include use of a device that diverts blood flow from other areas of the body to the brain. An example is a catheter partially occluding the aorta, such as the CoAxia NeuroFlo™ catheter device, which has recently been subjected to a randomized trial and may get FDA approval for stroke treatment. This device has been used on subjects presenting with stroke up to 14 hours after onset of ischemia.

Active agents of the invention including D amino acid(s) can be administered with any of the forms of reperfusion therapy to a subject amenable to treatment. However, the active agents of the invention are particularly advantageous for administration with thrombolytic agents because the inclusion of one or more D-amino acids in the active agent reduces the susceptibility of the active agent to cleavage by plasmin, which is induced by thrombolytic agents. Thus, the active agents including one or more D-amino acids can be co-administered with thrombolytic agents in which regimes, which would otherwise result in cleavage of the active agent induced by the thrombolytic agent. For example, the thrombolytic agent can be administered within a window of 60, 30, or 15 minutes before the active agent. In some methods, the active agent is administered at the same time as the thrombolytic agent. The active agent and thrombolytic agent can be co-formulated or administered separately. In some methods, the thrombolytic agent is administered before the active agent and persists at a detectable level in the serum when the active agent is administered.

For treatment of ischemias that cannot be predicted in advance, an active agent can be administered as soon as possible or practical after onset of ischemia. For example, an active agent can be administered within a period of 0.5, 1, 2, 3, 4, 5, 6, 9, 12 or 24 hours after the onset of ischemia. For ischemias that can be predicted in advance, an active agent can be administered before, concurrent with or after onset of ischemia. For example, for an ischemia resulting from surgery, the PDS-95 inhibitor is sometimes routinely administered in a period starting 30 minutes before beginning surgery and ending 1, 2, 3, 4, 5, 6, 9, 12 or 24 hours after surgery without regard to whether ischemia has or will develop. Because the active agents are free of serious side effects, they can be administered when stroke or other ischemic conditions are suspected without a diagnosis according to art-recognized criteria having been made. For example, an active agent can be administered at the location where the stroke has occurred (e.g., in the patients' home) or in an ambulance transporting a subject to a hospital. An active agent can also be safely administered to a subject at risk of stroke or other ischemic conditions before onset who may or may not actually develop the condition.

Following, or sometimes before, administration of an active agent, a subject presenting with sign(s) and/or symptom(s) of ischemia can be subject to further diagnostic assessment to determine whether the subject has ischemia within or otherwise affecting the CNS and determine whether the subject has or is susceptible to hemorrhage. Most particularly in subjects presenting with symptoms of stroke, testing attempts to distinguish whether the stroke is the result of hemorrhage or ischemia, hemorrhage accounting for about 17% of strokes. Diagnostic tests can include a scan of one or more organs, such as a CAT scan, MRI or PET imaging scan or a blood test for a biomarker that suggests that a stroke has occurred. Several biomarkers associated with stroke are known including B-type neurotrophic growth factor, von Willebrand factor, matrix metalloproteinase-9, and monocyte chemotactic protein-1 (see Reynolds et al., Clinical Chemistry 49: 1733-1739 (2003)). The organ(s) scanned include any suspected as being the site of ischemia (e.g., brain, heart, limbs, spine, lungs, kidney, retina) as well as any otherwise suspect of being the source of a hemorrhage. A scan of the brain is the usual procedure for distinguishing between ischemic and hemorrhagic stroke. Diagnostic assessment can also include taking or reviewing a subject's medical history and performing other tests. Presence of any of the following factors alone or in combination can be used in assessing whether reperfusion therapy presents an unacceptable risk: subject's symptoms are minor or rapidly improving, subject had seizure at onset of stroke, subject has had another stroke or serious head trauma within the past 3 months, subject had major surgery within the last 14 days, subject has known history of intracranial hemorrhage, subject has sustained systolic blood pressure >185 mmHg, subject has sustained diastolic blood pressure >110 mmHg, aggressive treatment is necessary to lower the subject's blood pressure, subject has symptoms suggestive of subarachnoid hemorrhage, subject has had gastrointestinal or urinary tract hemorrhage within the last 21 days, subject has had arterial puncture at noncompressible site within the last 7 days, subject has received heparin with the last 48 hours and has elevated PTT, subject's prothrombin time (PT) is >15 seconds, subject's platelet count is <100,000/μL. subject's serum glucose is <50 mg/dL or >400 mg/dL, subject is a hemophiliac or has other clotting deficiencies.

The further diagnostic investigation determines according to recognized criteria or at least with greater probability that before the investigation whether the subject has an ischemic condition, and whether the subject has a hemorrhage, has an unacceptable risk of hemorrhage or is otherwise excluded from receiving reperfusion therapy due to unacceptable risk of side effects. Subjects in which a diagnosis of an ischemic condition within or otherwise likely to affect the CNS is confirmed who are without unacceptable risk of side effects can then be subject to reperfusion therapy. Reperfusion therapy can be performed as soon as practical after completion of any diagnostic procedures.

Both treatment with an active agent and reperfusion therapy independently have ability to reduce infarction size and functional deficits due to ischemia. When used in combination according to the present methods, the reduction in infarction size and/or functional deficits is preferably greater than that front use of either agent alone administered under a comparable regime other than for the combination (i.e., co-operative). More preferably, the reduction in infarction side and/or functional deficits is at least additive or preferably more than additive (i.e., synergistic) of reductions achieved by the agents alone under a comparable regime except for the combination. In some regimes, the reperfusion therapy is effective in reducing infarction size and/or functional times at a time post onset of ischemia (e.g., more than 4.5 hr) when it would be ineffective but for the concurrent or prior administration of the PSD-95 inhibitor. Put another way, when a subject is administered an active agent and reperfusion therapy, the reperfusion therapy is preferably at least as effective as it would be if administered at an earlier time without the active agent. Thus, the active agent effectively increases the efficacy of the reperfusion therapy by reducing one or more damaging effects of ischemia before or as reperfusion therapy takes effect. The active agent can thus compensate for delay in administering the reperfusion therapy whether the delay be from delay in the subject recognizing the danger of his or her initial symptoms delays in transporting a subject to a hospital or other medical institution or delays in performing diagnostic procedures to establish presence of ischemia and/or absence of hemorrhage or unacceptable risk thereof. Statistically significant combined effects of an active agent and reperfusion therapy including additive or synergistic effects can be demonstrated between populations in a clinical trial or between populations of animal models in preclinical work.

Although the invention has been described in detail for purposes of clarity of understanding, certain modifications may be practiced within the scope of the appended claims. All publications, accession numbers, and patent documents cited in this application are hereby incorporated by reference in their entirety for all purposes to the same extent as if each were so individually denoted. To the extent more than one sequence is associated with an accession number at different times, the sequences associated with the accession number as of the effective filing date of this application is meant. The effective filing date is the date of the earliest priority application disclosing the accession number in question. Unless otherwise apparent from the context any element, embodiment, step, feature or aspect of the invention can be performed in combination with any other.

### Examples

The examples refer to peptides having the following names and sequences. Lower case letters indicate D-amino acids and upper case letters L-amino acids.

NA-1 (aka Tat-NR2B9c or nerinetide)
(SEQ ID NO: 58)
YGRKKRRQRRRKLSSIESDV
D-TAT-L-2B9c
(SEQ ID NO: 80)
ygrkkrrqrrrKLSSIESDV
NA-3
(SEQ ID NO: 6)
ygrkkrrqrrrklsslESDV
D-NA-1
(SEQ ID NO: 81)
ygrkkrrqrrrklssiesdv

1. Plasmin Cleavage Sites in NA-1

Plasmin is a serum protease induced by thrombolytic agents, such as tPA. Plasmin cleavage sites can occur on the C-terminal side of basic amino acids residues in a peptide formed of L-amino acids.

NA-1 was digested with plasmin and the products analyzed by mass spectrometry. The following cleavage products were detected

(SEQ ID NO: 58)
YGRKKRRQRRRKLSSIESDV (Full-length NA-1, undigested)
(SEQ ID NO: 82)
RRQRRRKLSSIESDV
(SEQ ID NO: 83)
RQRRRKLSSIESDV
(SEQ ID NO: 84)
QRRRKLSSIESDV
(SEQ ID NO: 85)
RRKLSSIESDV
(SEQ ID NO: 86)
RKLSSIESDV
(SEQ ID NO: 2)
KLSSIESDV
(SEQ ID NO: 87)
LSSIESDV

These cleavage products imply that NA-1 is subject to cleavage at seven of nine potential sites as shown in FIG. 1. However, cleavage at the other two sites may occur to a lesser extent.

2. Degradation of NA-1 Administered Simultaneously with tPA in Rat or Human Plasma

Rat or human plasma was treated with NA-1 alone or with recombinant tPA at the following concentrations:
NA-1 Alone [65 ug/mL] (N=4)
NA-1 [65 ug/mL]+rt-PA [22.5 ug/mL] (N=4)
NA-1 [65 ug/mL]+rt-PA [67.5 ug/mL] (N=4)
NA-1 [65 ug/mL]+rt-PA [135 ug/mL] (N=4)

Samples were collected at 6 different time points.

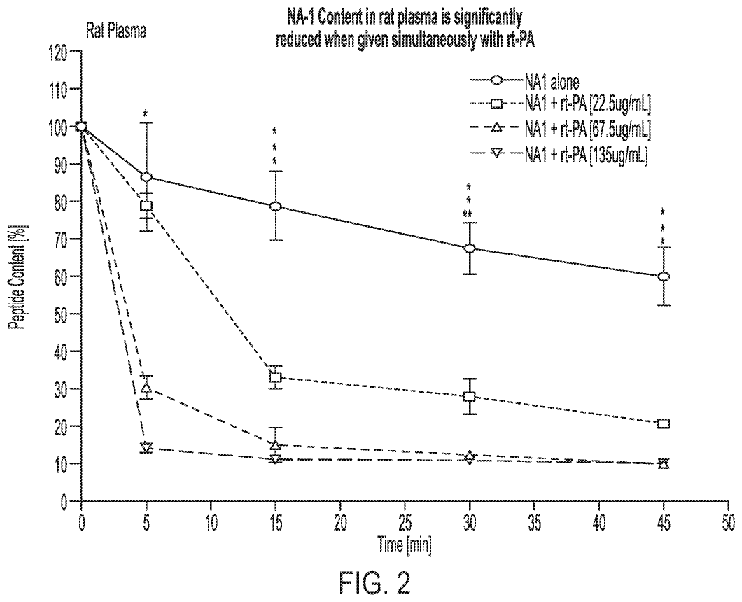

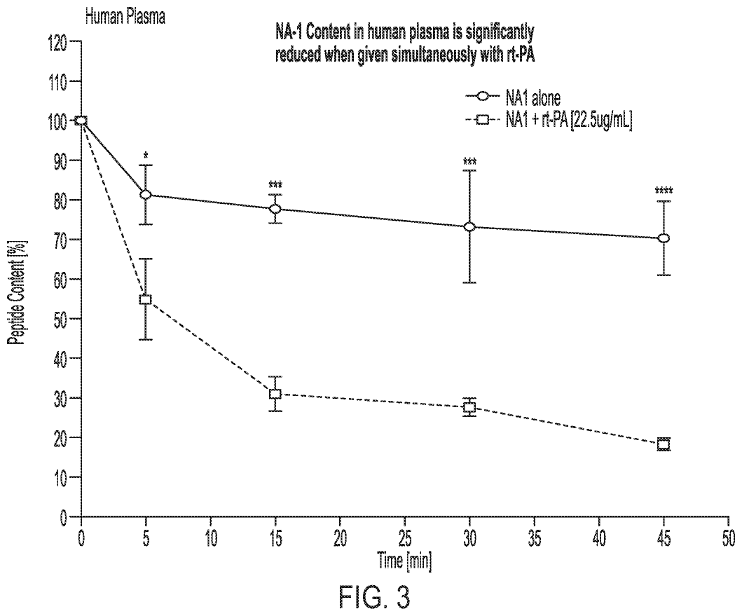

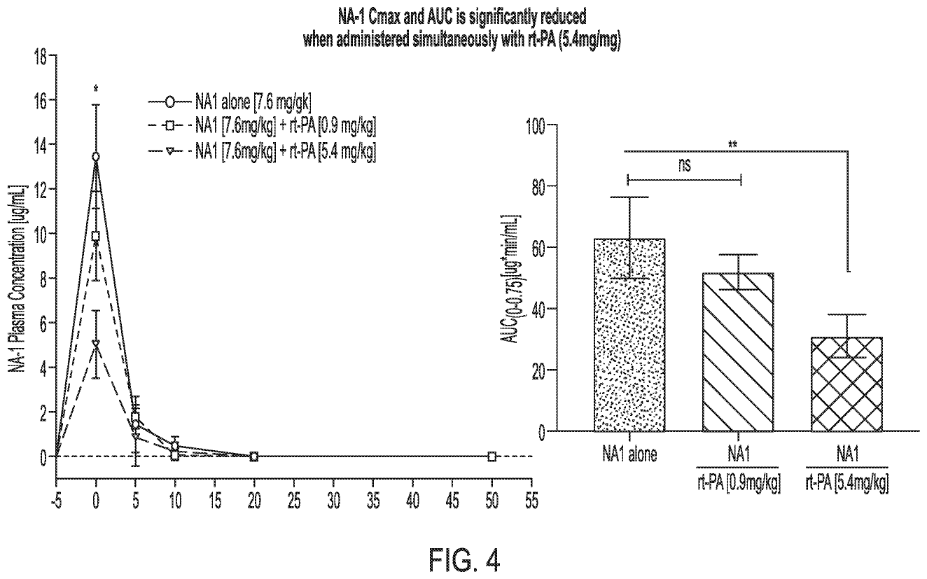

FIGS. 2 and 3 shows that NA-1 content decayed much more rapidly when tPA was co-administered than for NA-1 alone and rat plasma in vitro or human plasma in vitro, respectively. FIG. 4 shows a similar reduction in CMax and AUC after administering NA-1 and tPA to rats and collecting plasma to determine the NA-1 levels after various timepoints. Thus, tPA induces cleavages of NA-1 in rat or human plasma when the two are administered together in vitro or in vivo. Neither tPA nor TNK directly cleaves NA-1 in phosphate buffered saline alone (data not shown). Therefor the cleavage of NA-1 is a result of plasminogen activation in the context of plasma or blood in an animal.

3. Degradation of Peptides Including D-Amino Acids

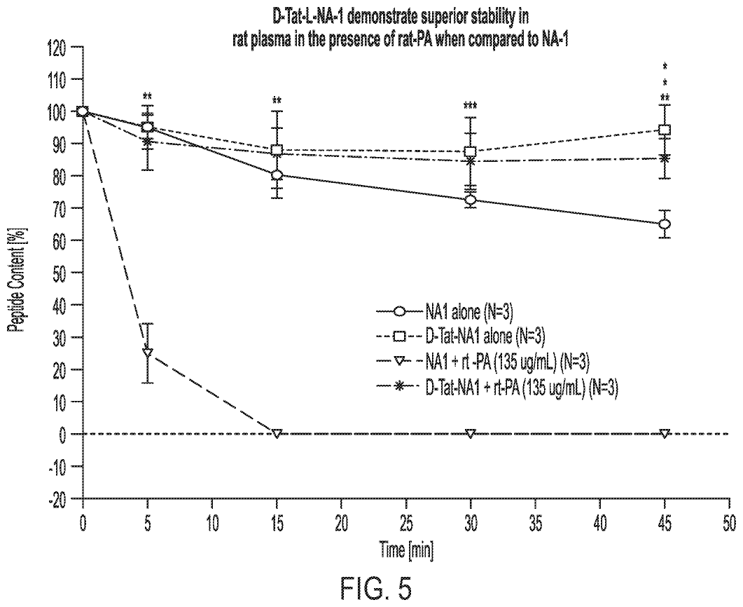

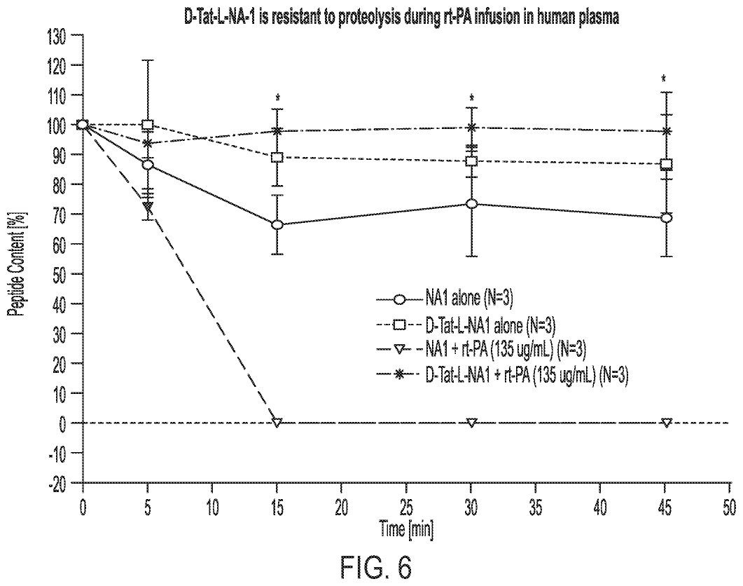

FIG. 5 compares NA-1 and D-Tat-L-2B9C (also called D-Tat-L-NA-1) alone or with tPA administered simultaneously in rat plasma in vitro. Whereas NA-1 treated with tPA decayed to zero within about 15 min, D-Tat-L-2B9C showed only negligible degradation when co-administered with tPA. FIG. 6 shows similar results with human plasma as rat plasma. Such degradation as occurred increased with the dose of tPA.

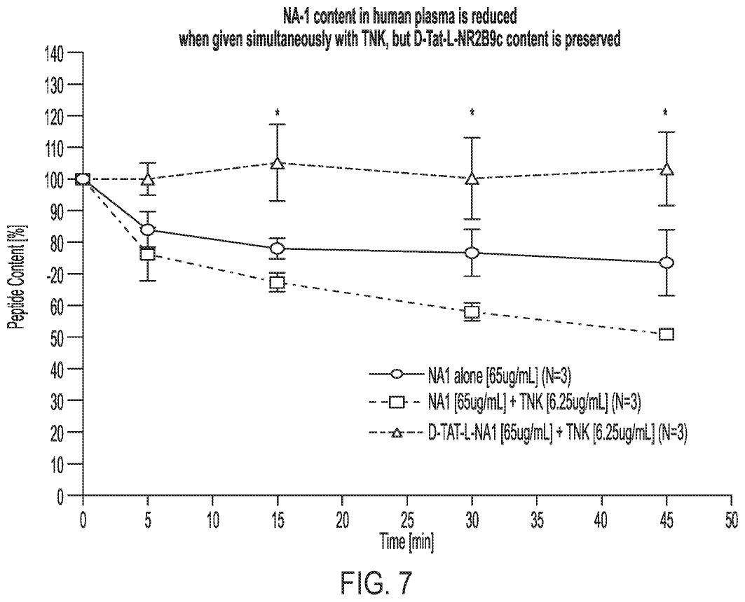

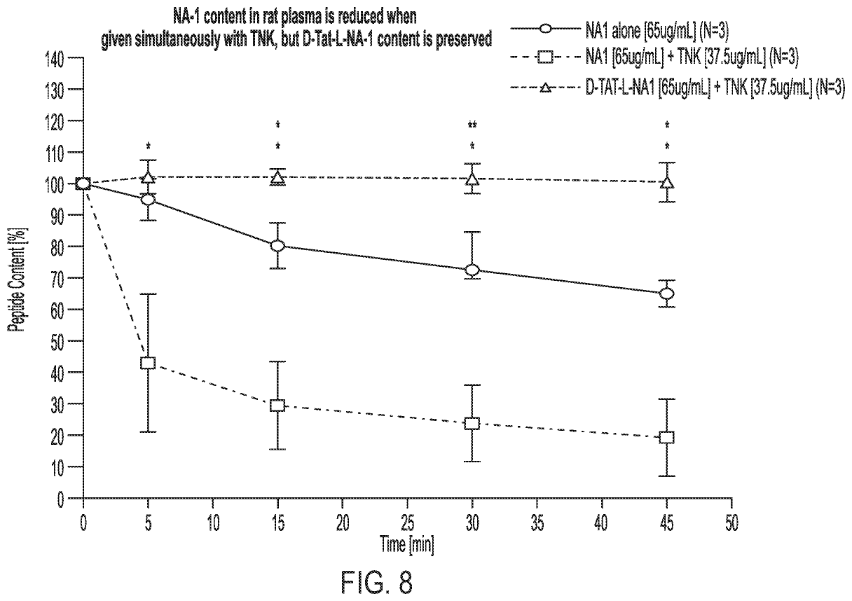

The experiment was repeated using TNK-tissue plasminogen activator in place of tPA. TNK-tissue plasminogen activator is a bioengineered variant of tPA having a longer half-life. Similar results were obtained with TNK as tPA. NA-1 showed rapid degradation with coadministration of TNK whereas D-Tat-L-2B9C was stable (FIGS. 7 and 8).

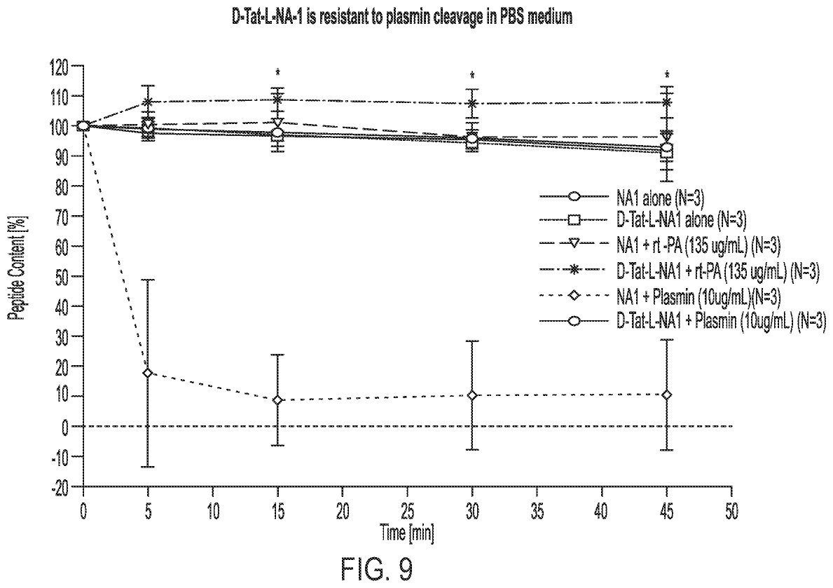

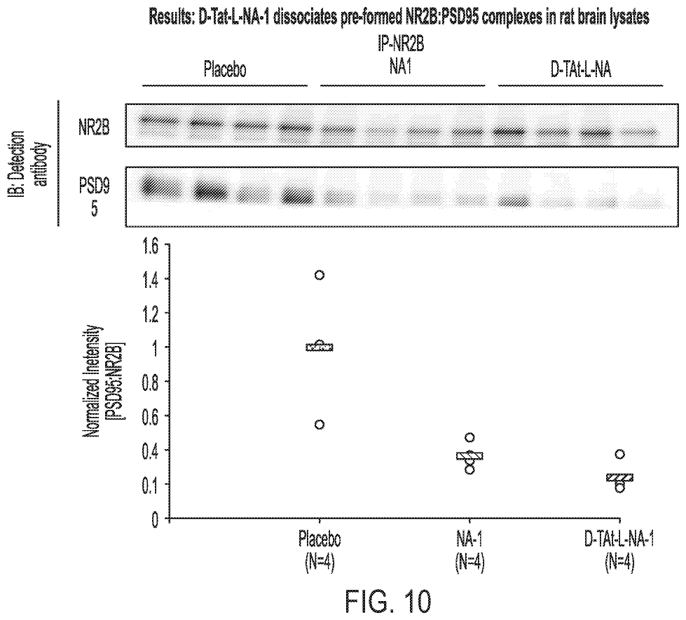

FIG. 9 shows similar results for treatment of NA-1 or D-Tat-L-2B9C with plasmin in PBS. NA-1 was rapidly degraded, whereas D-Tat-L-2B9C showed similar stability with or without plasmin. A control treatment with tPA in PBS buffer (no plasma) showed no degradation of either NA-1 or D-Tat-L-2B9C because without supplying plasma, tPA does not generate plasmin.

4. D-Tat-L-NR2B9c Disrupts PSD-95:NR2B9c Complexes

Sprague-Dawley rats were subject to three pial vessel model (3PVo). The rats were dosed 1 hr after stroke onset with placebo, NA-1 or D-Tat-L-2B9C, each at 7.6 mg/kg. Brains were harvested 2 h after stroke onsets. Cortical stroke areas were collected for analysis. Immunoprecipitations were performed with anti-PSD-95 or anti-NMDAR2B. The amount of PSD-95 and NMDAR2B in samples was analyzed by Western blotting. Reduction in PSD-95-NMDAR2B complex formation was assessed by fold decrease of placebo versus treatment. FIG. 10 shows that NA-1 and D-Tat-L-2B9C were both able to dissociate preformed NMDAR2B:PSD-95 complexes and work effectively in vivo.

5. Binding Affinity to PSD-95

Binding was evaluated with a competitive ELISA assay. A plate was coated with 1 ug/ml PSD95_{PDZ2} in 50 mM bicarbonate buffer overnight at 4C. The plate was blocked in 2% BSA in PBST (0.05%) for 2 h at room temperature. Then, we incubated the plate with the mixture of 150 ng/ml of biotinylated-NA-1 and the different test compounds at concentrations starting from 120 ug/ml in a 3-fold dilution overnight at 4C, after proper washing with PBS-T, the plate was incubated with (1:3000) SA-HRP for 30 min. The wells were washed again, and then incubated with TMB solution for 10 min. The reaction was stopped with 100 ul H₂SO₄. Absorbance was determined at 450 nm with the synergy H1 reader.

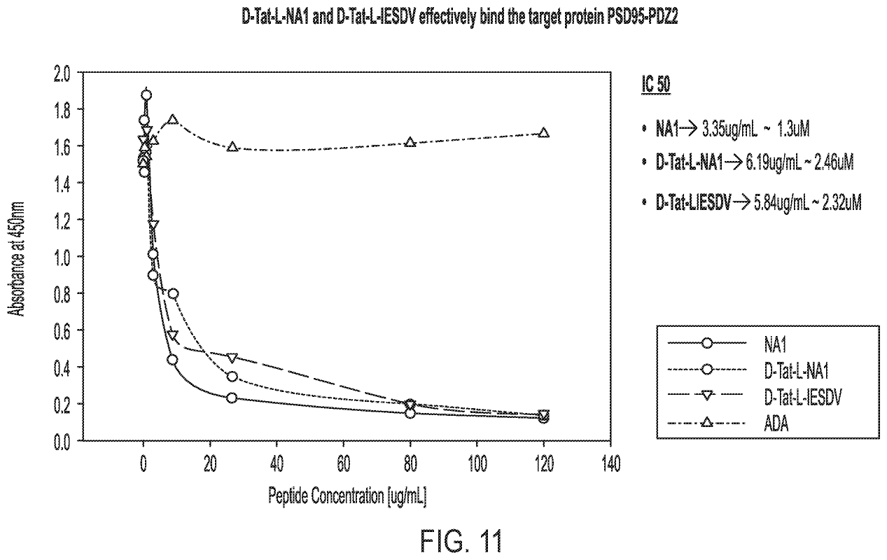

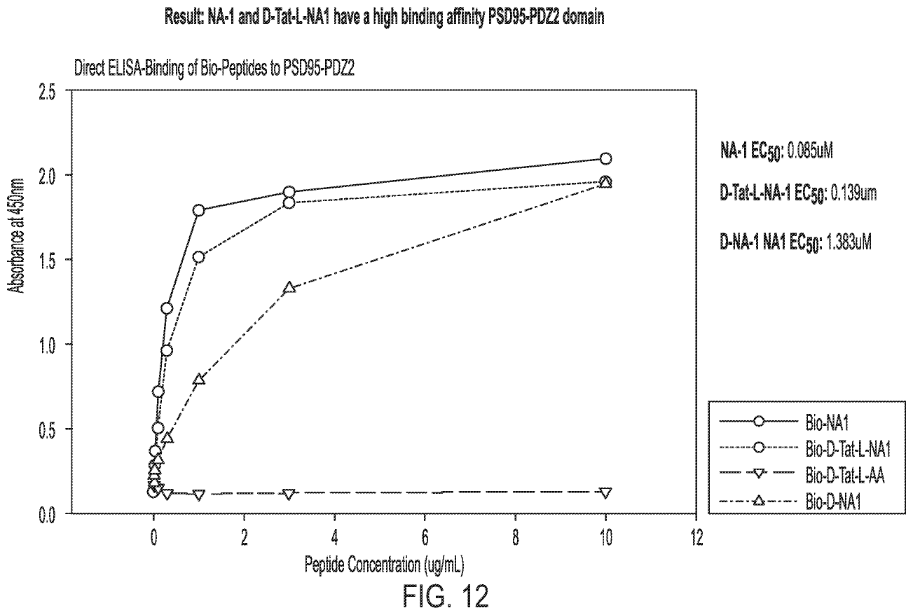

FIG. 12 shows that biotinylated NA-1, D-Tat-L-2B9C and D-Tat-L-IESDV (SEQ ID NO:6) each bound to PSD-95 domain 2 and shows EC50's for NA-1, D-Tat-L-2B9C and D-NA-1. The EC50's of NA-1 and D-Tat-L-2B9C were about the same within experimental error, whereas that of D-NA-1 was about ten-fold lower. This result provides evidence that converting C-terminal residues of NA-1 most responsible for binding to PSD-95 to D-amino acids reduces binding affinity. D-Tat-L-2B9C and D-Tat-L-IESDV (SEQ ID NO:6) effectively bind the target protein PS95_{PDZ2} in a dose-dependent manner. Both test peptides achieve IC₅₀ values <5 uM (FIG. 11). FIG. 11 shows the IC50's were within a factor of two of each other, which was within the margin of error of the experiment.

6. Pharmacokinetic Analysis

Rats were anaesthetized in the supine position (Isoflurane 1.5-%) and allowed to breath spontaneously in 0.5 L/min 02. The left femoral artery was cannulated for blood sampling.

Test agents were prepared at the stablish concentration in a total volume of vehicle. Pulmonary instillation was performed by intubation with a 14G catheter connected to a 1 cc syringe and the test agent will be delivered through the catheter. Subcutaneous (SQ or SC) injection was injected into the area of the left flank, no more than 2 ml of total volume per site.

The following compounds were tested: NA-1, D-Tat-L-NA1, D-Tat-L-IESDV (SEQ ID NO:6) and D-NA-1. Each dose was evaluated in 3 rats for each administration strategy. Planned dose levels and routes are indicated in the below Table 3. For the first experiment, evaluating two different administration routes (SQ and PI), blood samples were collected at 8 different timepoints: Pre and at 7 additional times (1, 2.5, 5, 10, 15, 30, 60 min) post dose (250 ul/sample). For the 24-hour PK curve experiment, blood samples were collected at 11 timepoints: Pre, 2.5, 5, 10, 15, 30, 60 min, 3 hr, 6 hr, 12 hr and 24 hr.

TABLE 4

| Delivery method | Compound | Dose (mg/kg) |
|---|---|---|
| Subcutaneous | NA-1 | 25 |
| | D-Tat-NR2B9c | 25 |
| | NA-3 | 25, 8.3, 2.8 |
| | D-NA-1 | 25 |
| Pulmonary Instillation | NA-1 | 25 |
| | D-Tat-NR2B9c | 25 |
| | NA-3 | 25 |
| | D-NA-1 | 25 |

HPLC quantification: Plasma was separated from blood and stored at −80° C. until used. Each sample was precipitated by adding 1M HCl (10 ul/100 ul sample) at >80° C., centrifuged (12,000 rpm×15 min) and the precipitate collected. A 5 cm C—18 RP-HPLC column was equilibrated with 10% acetonitrile with 0.1% TFA at 40° C., the sample was injected and run in an Agilent 1260 Infinity Quaternary LC System. (30 min at 1.5 mL/min; gradient from 10% to 35% acetonitrile in 0.1% TFA; Absorbance detected at 220 nm). Standard curves for HPLC were generated from plasma samples spiked with known quantities of test agent.

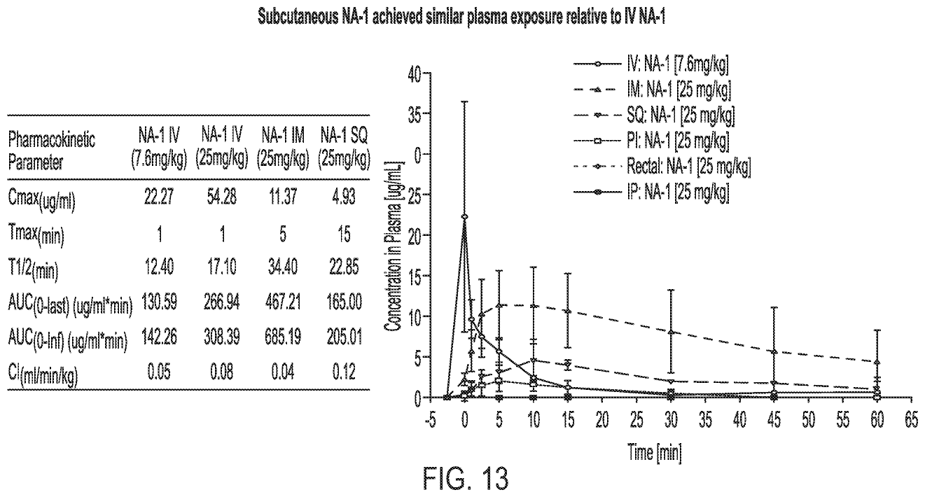

FIG. 13 shows that subcutaneous NA-1 had a much lower CMax and somewhat lower AUC than the same dose of intravenous NA-1 but has a longer half-life. Intramuscular NA-1 had a lower CMax, somewhat higher AUC and higher half-life than intravenous NA.

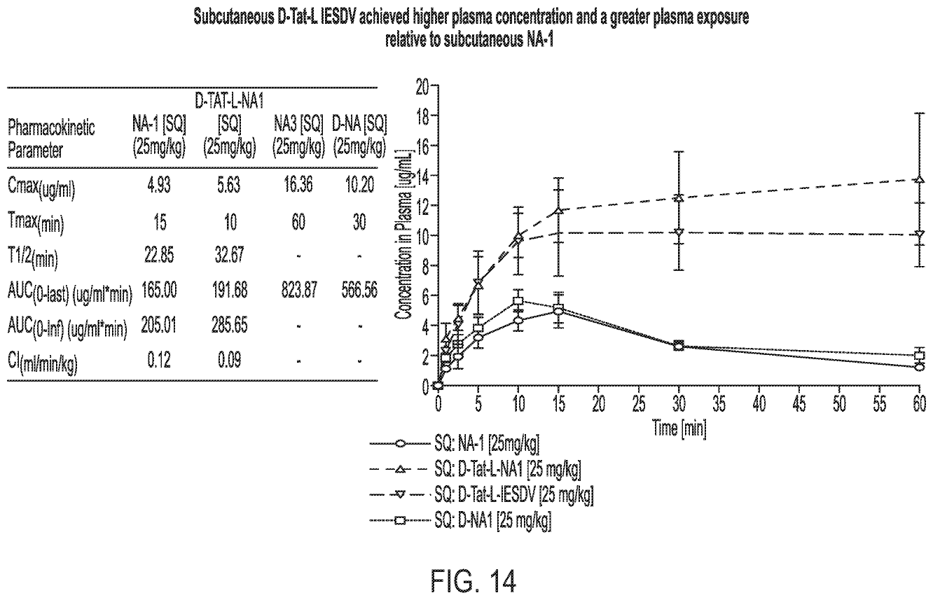

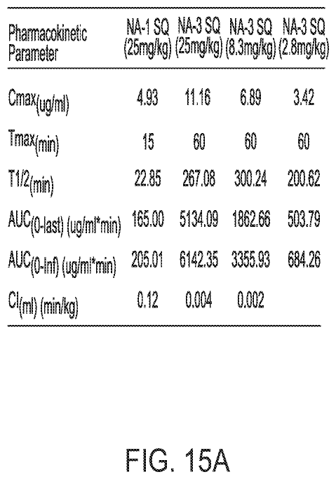

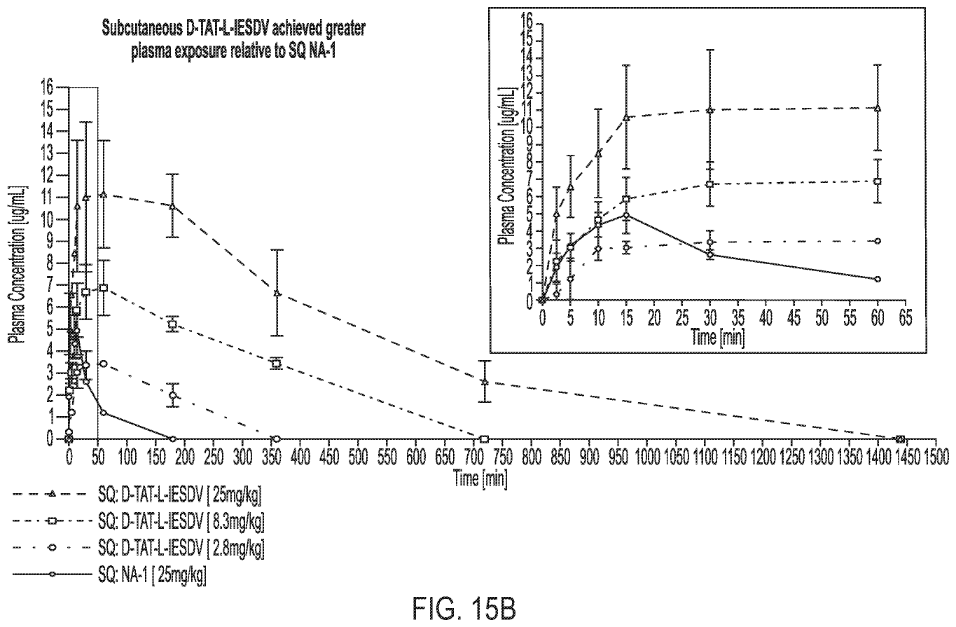

FIG. 14 shows that subcutaneous D-Tat-L-IESDV (SEQ ID NO:6) (NA-3) increased Cmax and AUC compared with subcutaneous NA-1. Subcutaneous D-Tat-L-2B9C and D-NA also increased Cmax and AUC relative to subcutaneous NA-1 but not to the same extent as D-Tat-L-IESDV (SEQ ID NO:6). FIGS. 15 A-B show that the Cmax and AUC of subcutaneous D-Tat-L-IESDV (SEQ ID NO:6) are dose-dependent increasing linearly with dose.

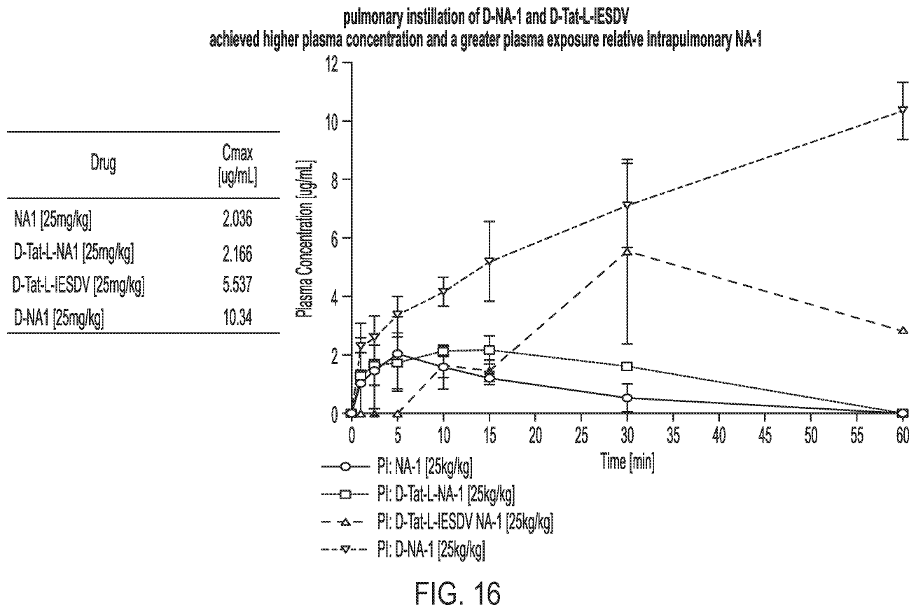

FIG. 16 shows that pulmonary instillation of D-Tat-L-IESDV (SEQ ID NO:6) resulted in a higher CMax than for NA-1 or D-Tat-L-2B9C.

7. Effect of Peptides on Histamine Release

The effect of D-Tat-L-IESDV (SEQ ID NO:6) injection on histamine release was tested in plasma samples from rats subjected to NA-3 [SQ] administration at three different doses. Blood samples were collected at 11 timepoints: Pre, 2.5, 5, 10, 15, 30, 60 min, 3 hr, 6 hr, 12 hr and 24 hr. Histamine levels were quantified by using commercially available histamine ELISA assay kit (Histamine ELISA-H1531-K01, Eagle Bioscience). The plates were coated with the plasma samples (50 ul/well) incubated for 60 minutes at room temperature on an orbital shaker with medium frequency. Then 100 ul of enzyme conjugate was added to the wells and incubated for 20 minutes at room temperature. Samples were washed again, and then incubated for 25 minutes at room temperature with TMB solution. The reaction was stopped with 100 ul H2SO4. Absorbance was determined on an ELISA plate reader at 450 nm.

Blood samples were taken at pre-injection and at 0, 1, 2.5, 5, 10, 15, 30- and 60-minutes post dose and used for histamine levels quantification using commercially available kit. This sampling period covers the period of histamine elevation observed for NA-1 after IV injection in rats' samples (N=3 animals/group).

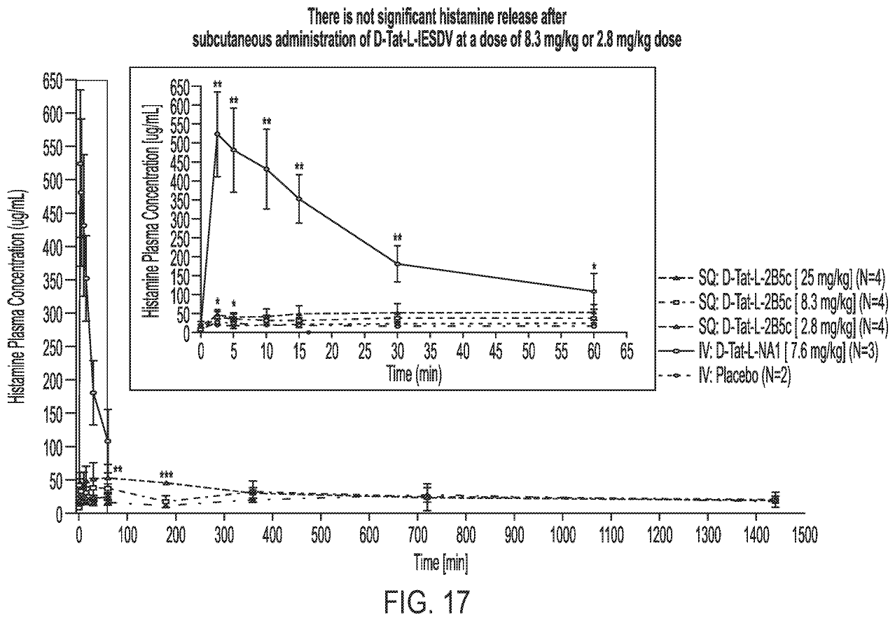

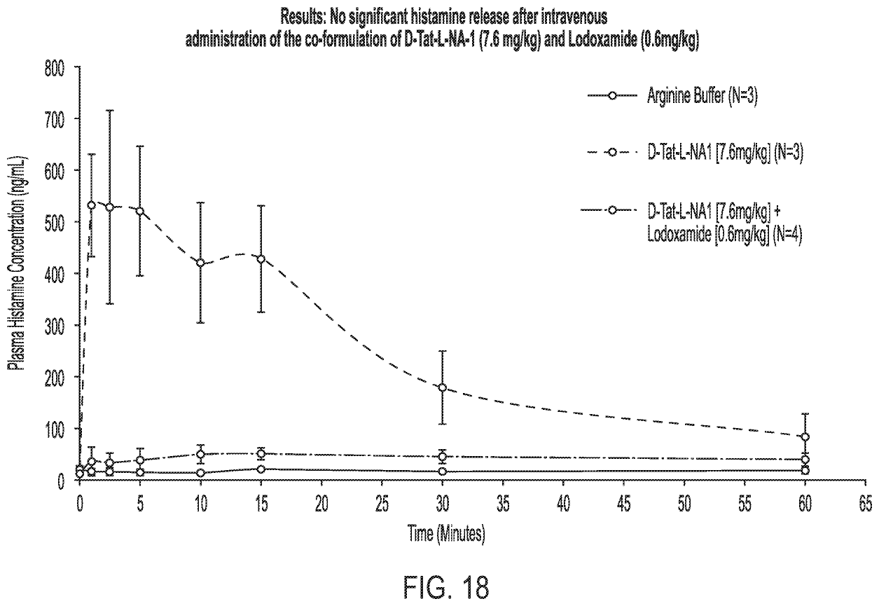

FIG. 17 shows that subcutaneous administration of D-Tat-L-IESDV (SEQ ID NO:6) at a dose of 8.3 mg/kg or 2.8 mg/kg did not result in significant histamine release. Intravenous D-Tat-L-NA1 at 7.6 mg/kg IV did result in significant histamine release. D-Tat-L-IESDV (SEQ ID NO:6) at 25 mg/kg SQ resulted in histamine release but still much less than 7.6 mg/kg IV. The histamine induced by D-Tat-L-2B9C at 7.6 mg/kg IV was abrogated by co-administration of lodoxamide (FIG. 18).

Therefore subcutaneous administration of active agents including D-amino acids results in reduced histamine release and at higher dosages than for intravenous administration.

8. Efficacy of D-Tat-L-2B9C as a Neuroprotectant in an Embolic MCA Occlusion Model

The animals were anesthetized with isoflurane (5% for induction, 2% for surgery, and 1.5% for maintenance. The femoral veins and arteries were cannulated with PE-50 tubing for drug administration, blood pressure monitoring and blood sampling. A complete monitoring (Cerebral blood flow, Arterial blood gases, Plasma glucose, Temperature) will be performed before and during the surgery. All physiological parameters will be maintained within normal range and relative cerebral blood flow was continuously measure with a PR407-1 straight needle LDF-probe (Perimed, Järfälla, Stockholm, Sweden) was connected to a standard laser Doppler monitor (PF5010 LDPM Unit and PF5001 main unit, Perimed, Järfälla, Stockholm, Sweden). For the middle cerebral artery embolic stroke, PE-50 tubing with a PE-10 5 cm tip was inserted via the external carotid artery into the internal carotid artery to the skull base and a previously prepared single red blood clot will be injected manually. After 7 minutes, the catheter and the clip on the common carotid artery (CCA) was removed. Animals were kept under anesthesia for the whole procedure and injection. Treatment drugs were administered simultaneously 1 hour after stroke onset. Neuroprotectants were injected as an intravenous bolus (<30 sec) and thrombolytic agents were administered as an initial 10% bolus injection in 1 min and the remaining 90% of the total dose as an infusion over 1 hour. After administration was finished, the animals were recovered in a clean cage with a heating lamp. Due to the acute nature of this stroke model, we only performed the neurological score test (postural reflex and forelimb placing tests (Grading from 0-12) as a behavioral assessment. Immediately after the neuroscore test (24 hours after stroke onset), the animals were euthanized. The brain was removed and sectioned coronally into 8 slices 1.5 mm thick, placed in a 2% solution of 2,3,5-triphenyltetrazolium chloride (TTC) at 37 degree Celsius for staining. The sections were scanned, and infarct volume measured with ImageJ software. Brain swelling was measured as well.

The study included the following groups:
Sham (no surgery) (N=10)
Placebo (negative control) (N=12)
NA-1 alone [7.6 mg/kg] (positive control) (N=11)
D-TAT-L-NA1_{Lodo} [7.6 mg/kg] (N=12)
rt-PA alone [5.4 mg/kg] (N=10)
NA-1 [7.6 mg/kg]+rt-PA [5.4 mg/kg] (negative control) (N=12)
D-TAT-L-NA1_{Lodo} [7.6 mg/kg]+rt-PA [5.4 mg/kg] (N=17)

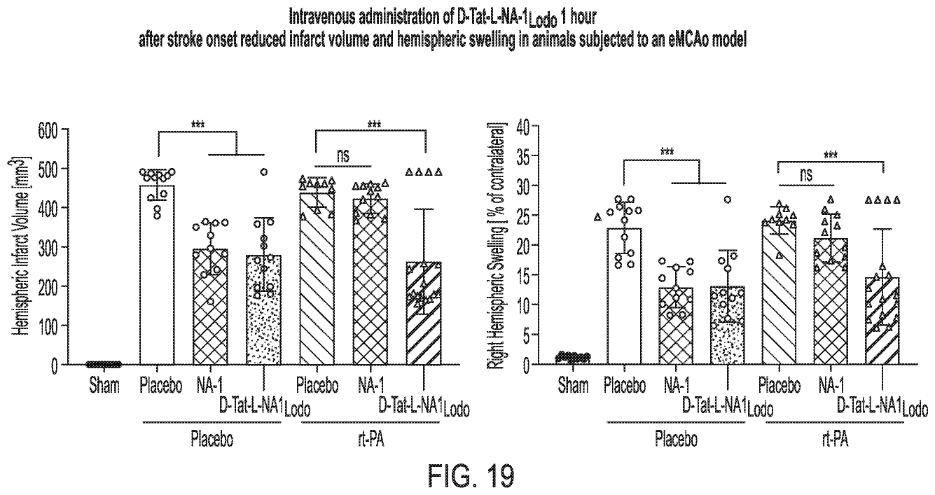

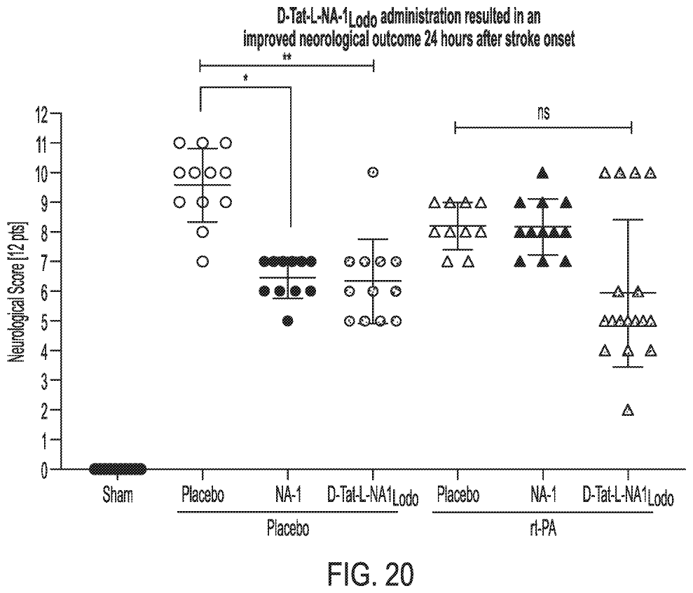

FIG. 19 shows that without tPA treatment, NA-1 and D-Tat-L-2B9C plus lodoxamide both significantly reduced infarction volume and right hemisphere swelling. When tPA was co-administered only the D-Tat-L-NA1 lodoxamide combination significantly protected against infarction and right hemisphere swelling. This result can be explained by tPA-induced proteolysis of NA-1 reducing its effect. D-Tat-L-NA1 is protected against such proteolysis by inclusion of D residues, so is still effective. FIG. 20 shows similar results for neurological outcome. Therefore, D-Tat-L-2B9C shows an improvement in plasma stability which translates in a reduction in stroke volume and improved behavioral outcome even when simultaneously administered with a thrombolytic agent such as rt-PA.

9. Subcutaneous Administration of PSD-95 Inhibitors

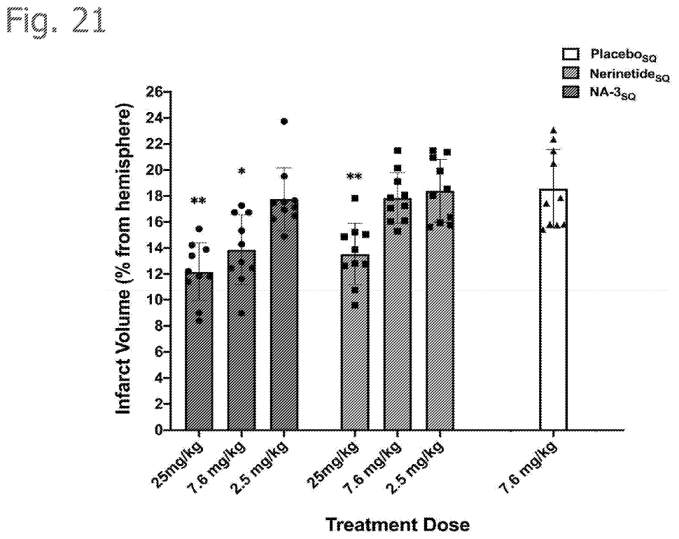

To demonstrate that PSD-95 inhibitors containing D amino acids other than the C-terminal 5 amino acids of the inhibitor would both be effective in models of stroke and able to be administered as a subcutaneous injection, a series of animal experiments were performed. FIG. 21 compares subcutaneous administration of 3 dose levels (2.6, 7.6 or 25 mg/kg) of nerinetide or NA-3 in a rat 3-pial vessel occlusion model of stroke. Treatments were administered subcutaneously as a bolus injection, 60 minutes after the onset of stroke. Rats receiving NA-3 and nerinetide at a concentration of 25 mg/kg demonstrated a significant reduction in infarct volume when compared to placebo. NA-3 at 7.6 mg/kg had also a significant reduction in infarct volume, but nerinetide at the same concentration failed to achieved infarct volume reduction. Data is presented as mean±SD, N=10/group. The asterisk (*) represents statistical significance when compared to Placebo (ANOVA, with a Tukey's post-hoc analysis, *P<0.0332, **P<0.0021, ***P<0.0002 and ****P<0.0001). NA-3 was effective in this model, indicating that changing all of the amino acids to D amino acids other than the C-terminal 5 amino acids (IESDV, SEQ ID NO:5) is effective in stroke and PSD-95 inhibition. Further, the increased stability likely contributes to the improved efficacy over nerinetide when administered subcutaneously. Equivalent neuroprotection (reduction in infarct volume) is observed between the 25 mg/kg dose of nerinetide and the 7.6 mg/kg dose of NA-3, suggesting that a 3-fold lower dose of NA-3 is as effective.

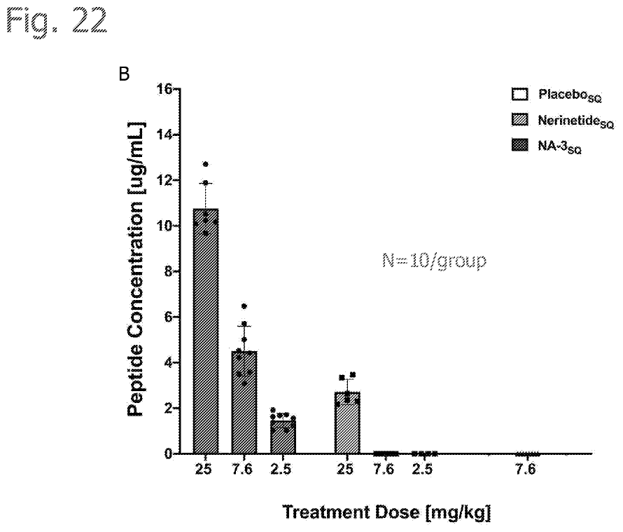

Unlike transient models of stroke in rats where much lower doses are required for neuroprotection, neuroprotection in 3 pial vessel occlusion models of stroke seems to require plasma concentrations of nerinetide at or above 2 ug/mL (or molar equivalent). FIG. 22 shows the plasma concentrations of NA-3 and nerinetide at 15 minutes post dose from the animals in the previous model (FIG. 21). Although the plasma levels continue to increase through about 3 hours then decrease, it is important to achieve rapid accumulation in the blood and brain for emergency indications like stroke. This demonstrates that subcutaneous administration of PSD-95 inhibitors of the structures presented herein can achieve therapeutic concentrations in a rapid timeframe. For pharmacokinetic sample analysis, calibration standard samples at concentrations of 0, 2.5, 5, 10, 15, 20 and 40 ug/mL of nerinetide were prepared by spiking 1 μL of the appropriate stock to 100 μL of plasma. For the NA-3 standard curve for pharmacokinetic sample analysis, calibration standard samples at concentrations of 0, 2.5, 5, 10, 15, 20 and 40 ug/mL of NA-3 were prepared by spiking 1 uL of the appropriate stock to 100 L of plasma. Blood samples were collected 15 min after subcutaneous administration of nerinetide at 25 mg/kg (N=6), nerinetide at 7.6 mg/kg (N=8), nerinetide at 2.5 mg/kg (N=4) or NA-3 at 25 mg/kg (N=7), NA-3 at 7.6 mg/kg (N=9), NA-3 at 2.5 mg/kg (N=9) and Placebo (N=8). Data is presented as mean±SD. This graph shows a dose proportionality between the treatment dose and the Cmax following single subcutaneous administration of either nerinetide or NA-3. NA-3 shows a higher stability in plasma and a higher concentration at 15 min post-dose when compared to nerinetide at the same dose.

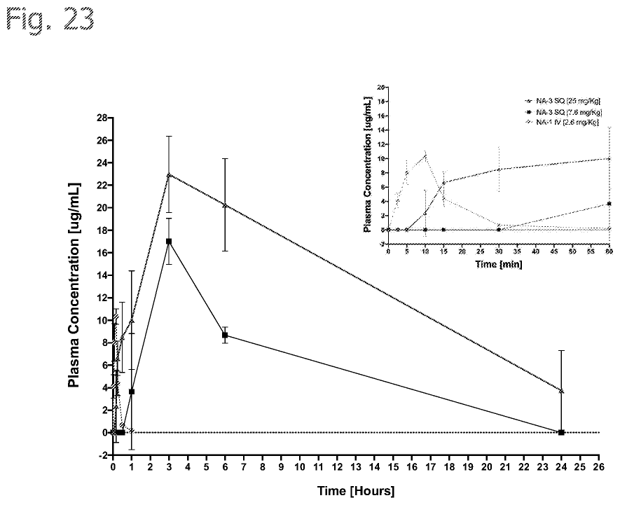

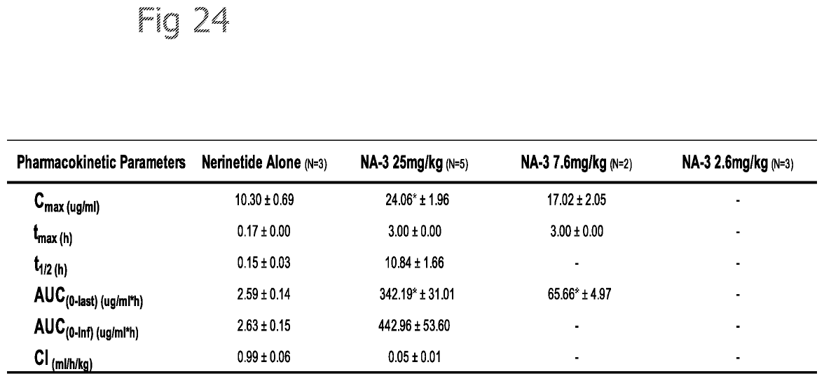

To confirm that plasma levels equivalent or greater than the known effective concentrations from the human studies (˜10 ug/mL plasma concentration for a 2.6 mg/kg dose), 25 mg/kg or 7.6 mg/Kg NA-3 was administered to non-human primates (cynomolgus macaques) as a subcutaneous injection and plasma samples were tested at different time points (FIG. 23). Both concentrations were able to achieve plasma concentrations higher than those demonstrated to be effective in humans and greater than an intravenous dose of 2.6 mg/kg NA-1 (Hill, Lancet 2020). FIG. 24 shows the pharmacokinetic profiles for the injection levels tested. All values are presented as mean±SD; Statistical significance when compared to nerinetide alone is indicated as * (one-way ANOVA with post-hoc turkey's correction, *P<0.01). (Cₘₐₓ: maximum plasma concentrations based on the extrapolated time-zero value; t_{1/2}: half-life at terminal phase; tₘₐₓ: time to reach Cmax; AUC₀₋ₜ: area under the concentration-time curve from 0 to the last measured value; AUC_{0-inf}: extrapolated area under the concentration-time curve from 0 to infinity; Cl: total clearance). Data is presented as mean±SD of three-four animals per group. The (−) represent data not reported due to an extrapolation of the AUC_{0-inf} greater than 20% and R² lower than 0.9.

What is claimed is:

1. An active agent comprising an internalization peptide linked to an inhibitor peptide, which inhibits PSD-95 binding to NOS and/or NMDAR2B, wherein the internalization peptide has an amino acid sequence comprising YGRKKRRQRRR (SEQ ID NO:1) and the inhibitor peptide has a sequence comprising KLSSIESDV (SEQ ID NO:2), or a variant thereof with up to five substitutions or deletions total in the internalization peptide and inhibitor peptide, wherein at least the four C-terminal amino acids of the inhibitor peptide are L-amino acids, and a contiguous segment of amino acids including all of the R and K residues are D-amino acids.

2. The active agent of claim 1, wherein the residue immediately C-terminal to the most C-terminal R or K residue is also a D-residue.

3. The active agent of claim 1 or 2, wherein the C-terminal of the internalization peptide is linked to the N-terminus of the inhibitor peptide as a fusion peptide.

4. The active agent of any preceding claim, wherein the inhibitor peptide comprises [E/D/N/Q]-[S/T]-[D/E/Q/N]-[V/L] (SEQ ID NO:3) at the C-terminus.

5. The active agent of any preceding claim, wherein the inhibitor peptide comprises I-E-[S/T]-D-V (SEQ ID NO:4) at the C-terminus.

6. The active agent of claim 1, wherein the inhibitor peptide comprises IESDV (SEQ ID NO:5) at the C-terminus.

7. The active agent of any preceding claim, wherein each of the five C-terminal amino acids of the inhibitor peptide are L-amino acids.

8. The active agent of claim 7, wherein every other residue of the active agent is a D-amino acid.

9. The active agent of claim 1 having the amino acid sequence ygrkkrrqrrrklsslESDV (SEQ ID NO:6), ygrkkrrqrrrksslESDV (SEQ ID NO:7), ygrkkrrqrrrkslESDV (SEQ ID NO:8), or ygrkkrrqrrrklESDV (SEQ ID NO:9).

10. The active agent of claim 1, having the amino acid sequences ygrkkrrqrrrklsslESDV (SEQ ID NO:6), wherein the lower case letter are D-amino acids and the upper case letters are L-amino acids.

11. The active agent of any preceding claim having enhanced stability in plasma compared with Tat-NR2B9c.

12. The active agent of any preceding claim having enhanced plasmin resistance compared with Tat-NR2B9c.

13. The active agent of any preceding claim having a binding affinity for PSD-95 within 2- fold of Tat-NR2B9c.

14. The active agent of any preceding claim having an IC50 for inhibiting PSD-95 binding to NMDAR2B within 2-fold of Tat-NR2B9c.

15. The active agent of any preceding claim, which competes with Tat-NR2B9c for binding to PSD-95.

16. The active agent of any preceding claim as a chloride salt.

17. A formulation of an active agent of any preceding claim, further comprising histidine and trehalose.

18. A formulation of an active agent of any of claims 1-16, further comprising a phosphate buffer.

19. A coformulation comprising the active agent of any preceding claim and an anti-inflammatory agent.

20. The co-formulation of claim 19, wherein the anti-inflammatory is a mast cell degranulation inhibitor or antihistamine.

21. A co-formulation comprising the active agent of any preceding claim and a thrombolytic agent.

22. A method of treating a subject having or at risk of a condition selected from stroke, cerebral ischemia, traumatic injury to the CNS, subarachnoid hemorrhage, pain, anxiety, epilepsy, comprising administering an effective regime of the active agent of any preceding claim to the subject.

23. A method of treating ischemic stroke in a subject having or at risk of stroke, comprising administering an effective regime of an active agent to the subject, wherein the subject is co-administered a thrombolytic agent, wherein the active agent comprises an internalization peptide linked to an inhibitor peptide, which inhibits PSD-95 binding to NOS and/or NMDAR2B, wherein at least the four C-terminal amino acids of the inhibitor peptide are L-amino acids, and at least one of the remaining amino acids of the active agent is a D-amino acid, wherein the active agent and thrombolytic agent are administered sufficiently proximate in time that cleavage of the active agent induced by the thrombolytic agent is reduced by the inclusion of the at least one D-amino acid.

24. The method of claim 23, wherein the internalization peptide is linked at its N-terminus to the C-terminus of the inhibitor peptide as a fusion protein.

25. The method of claim 23, wherein the inhibitor peptide comprises [E/D/N/Q]-[S/T]-[D/E/Q/N]-[V/L](SEQ ID NO:3) as the last four residues.

26. The method of claim 23, wherein the inhibitor peptide comprises [I]-[E/D/N/Q]-[S/T]-[D/E/Q/N]-[V/L] (SEQ ID NO:10) as the last five residues, each of which is an L amino acid.

27. The method of claim 23, wherein the internalization peptide is a tat peptide.

28. The method of claim 27, wherein at least 8 residues of the tat peptide are D-amino acids.

29. The method of claim 27, wherein each residue of the tat peptide is a D-amino acid.

30. The method of any preceding claim 23, comprising YGRKKRRQRRR (SEQ ID NO:1) as the internalization peptide linked at its N-terminus to KLSSIESDV (SEQ ID NO:2) or KLSSIETDV (SEQ ID NO:12) as the inhibitor peptide forming a fusion protein.

31. The method of claim 30, wherein the active agent comprises a contiguous segment of D-residues including each of the K and R residues.

32. The method of claim 23, wherein the active agent comprises ygrkkrrqrrrklsslESDV (SEQ ID NO:6), wherein lower case letters represent D-amino acids and upper case letters are L-amino acids.

33. The method of any preceding claim, wherein the thrombolytic agent is administered within a window of 60, 30 or 15 minutes before the active agent.

34. The method of any preceding claim, wherein the active agent and thrombolytic agent are administered at the same time.

35. A method of delivering an active agent to a subject in need thereof, comprising administering the active agent as defined in any preceding claim by a nonintravenous route, wherein the active agent is delivered to the plasma at a therapeutic level.

36. The method of claim 35, wherein the active agent is administered subcutaneously.

37. The method of claim 35, wherein the active agent is administered intramuscularly.

38. The method of claim 35, wherein the active agent is administered intranasally or intrapulmonarily.

39. The method of claim 35, wherein the dose is greater than 3 mg/kg.

40. The method of claim 35, wherein the dose is greater than 10 mg/kg.

41. The method of claim 35, wherein the dose is greater than 20 mg/kg.

42. The method of claim 35, wherein the dose is below 10 mg/kg and the active agent is administered without co-administration of a mast cell degranulating inhibitor or anti-histamine.

43. The method of claim 35, wherein the dose is above 10 mg/kg and the active agent is administered

45. The method of claim 35, wherein the subject has or is at risk of a condition selected from stroke, cerebral ischemia, traumatic injury to the CNS, pain, anxiety, epilepsy, subarachnoid hemorrhage, Alzheimer's disease or Parkinson's disease.


SEQUENCE LISTING (87 sequences)

SEQ ID NO: 1 - protein, 11 aa, Artificial sequence
  Synthesized
YGRKKRRQRRR

SEQ ID NO: 2 - protein, 9 aa, Artificial sequence
  Synthesized
KLSSIESDV

SEQ ID NO: 3 - protein, 4 aa, Artificial sequence
  Synthesized
  MISC_FEATURE (1)..(1): Xaa is Glu, Asp, Asn, or Gln
  MISC_FEATURE (2)..(2): Xaa is Ser or Thr
  MISC_FEATURE (3)..(3): Xaa is Asp, Glu, Gln, or Asn
  MISC_FEATURE (4)..(4): Xaa is Val or Leu
XXXX

SEQ ID NO: 4 - protein, 5 aa, Artificial sequence
  Synthesized
  MISC_FEATURE (3)..(3): Xaa is Ser or Thr
IEXDV

SEQ ID NO: 5 - protein, 5 aa, Artificial sequence
  Synthesized
IESDV

SEQ ID NO: 6 - protein, 20 aa, Artificial sequence
  Synthesized
  MISC_FEATURE (1)..(15): D-amino acids
YGRKKRRQRRRKLSSIESDV

SEQ ID NO: 7 - protein, 19 aa, Artificial sequence
  Synthesized
  MISC_FEATURE (1)..(14): D-amino acids
YGRKKRRQRRRKSSIESDV

SEQ ID NO: 8 - protein, 18 aa, Artificial sequence
  Synthesized
  MISC_FEATURE (1)..(13): D-amino acids
YGRKKRRQRRRKSIESDV

SEQ ID NO: 9 - protein, 17 aa, Artificial sequence
  Synthesized
  MISC_FEATURE (1)..(12): D-amino acids
YGRKKRRQRRRKIESDV

SEQ ID NO: 10 - protein, 5 aa, Artificial sequence
  Synthesized
  MISC_FEATURE (2)..(2): Xaa is Glu, Asp, Asn, or Gln
  MISC_FEATURE (3)..(3): Xaa is Ser or Thr
  MISC_FEATURE (4)..(4): Xaa is Asp, Glu, Gln, or Asn
  MISC_FEATURE (5)..(5): Xaa is Val or Leu
IXXXX

SEQ ID NO: 11 - protein, 10 aa, Artificial sequence
  Synthesized
GRKKRRQRRR

SEQ ID NO: 12 - protein, 9 aa, Artificial sequence
  Synthesized
KLSSIETDV

SEQ ID NO: 13 - protein, 9 aa, Artificial sequence
  Synthesized
RKKRRQRRR

SEQ ID NO: 14 - protein, 4 aa, Artificial sequence
  Synthesized
ESDV

SEQ ID NO: 15 - protein, 4 aa, Artificial sequence
  Synthesized
ESEV

SEQ ID NO: 16 - protein, 4 aa, Artificial sequence
  Synthesized
ETDV

SEQ ID NO: 17 - protein, 4 aa, Artificial sequence
  Synthesized
ETAV

SEQ ID NO: 18 - protein, 4 aa, Artificial sequence
  Synthesized
ETEV

SEQ ID NO: 19 - protein, 4 aa, Artificial sequence
  Synthesized
DTDV

SEQ ID NO: 20 - protein, 4 aa, Artificial sequence
  Synthesized
DTEV

SEQ ID NO: 21 - protein, 5 aa, Artificial sequence
  Synthesized
IESEV

SEQ ID NO: 22 - protein, 5 aa, Artificial sequence
  Synthesized
IETDV

SEQ ID NO: 23 - protein, 5 aa, Artificial sequence
  Synthesized
IETAV

SEQ ID NO: 24 - protein, 5 aa, Artificial sequence
  Synthesized
IETEV

SEQ ID NO: 25 - protein, 5 aa, Artificial sequence
  Synthesized
IDTDV

SEQ ID NO: 26 - protein, 5 aa, Artificial sequence
  Synthesized
IDTEV

SEQ ID NO: 27 - protein, 4 aa, Artificial sequence
  Synthesized
  MISC_FEATURE (1)..(1): Xaa is Glu, Gln, Ala, or an analog thereof
  MISC_FEATURE (2)..(2): Xaa is Thr or Ser
  MISC_FEATURE (3)..(3): Xaa is Ala, Gln, Asp, Asn, N-Me-Ala, N-Me-Gln, N-Me-Asp, N-Me-Asn, or an analog thereof
XXXV

SEQ ID NO: 28 - protein, 5 aa, Artificial sequence
  Synthesized
  MISC_FEATURE (2)..(2): Xaa is Glu, Gln, Ala, or an analog thereof
  MISC_FEATURE (3)..(3): Xaa is Thr or Ser
  MISC_FEATURE (4)..(4): Xaa is Ala, Gln, Asp, Asn, N-Me-Ala, N-Me-Gln, N-Me-Asp, N-Me-Asn, or an analog thereof
IXXXV

SEQ ID NO: 29 - protein, 14 aa, Artificial sequence
  Synthesized
RLSGMNEVLSFRWL

SEQ ID NO: 30 - protein, 5 aa, Artificial sequence
  Synthesized
GSSSS

SEQ ID NO: 31 - protein, 5 aa, Artificial sequence
  Synthesized
TGEKP

SEQ ID NO: 32 - protein, 8 aa, Artificial sequence
  Synthesized
GGRRGGGS

SEQ ID NO: 33 - protein, 9 aa, Artificial sequence
  Synthesized
LRQRDGERP

SEQ ID NO: 34 - protein, 13 aa, Artificial sequence
  Synthesized
YGRKKRRQRRRPQ

SEQ ID NO: 35 - protein, 12 aa, Artificial sequence
  Synthesized
GRKKRRQRRRPQ

SEQ ID NO: 36 - protein, 11 aa, Artificial sequence
  Synthesized
GRKKRRQRRRP

SEQ ID NO: 37 - protein, 11 aa, Artificial sequence
  Synthesized
  MISC_FEATURE (1)..(1): Xaa is an amino acid other than Tyr
XGRKKRRQRRR

SEQ ID NO: 38 - protein, 11 aa, Artificial sequence
  Synthesized
FGRKKRRQRRR

SEQ ID NO: 39 - protein, 10 aa, Artificial sequence
  Synthesized
RRRQRRKKRG

SEQ ID NO: 40 - protein, 11 aa, Artificial sequence
  Synthesized
RRRQRRKKRGY

SEQ ID NO: 41 - protein, 10 aa, Artificial sequence
  Synthesized
GKKKKKQKKK

SEQ ID NO: 42 - protein, 10 aa, Artificial sequence
  Synthesized
GAKKRRQRRR

SEQ ID NO: 43 - protein, 9 aa, Artificial sequence
  Synthesized
AKKRRQRRR

SEQ ID NO: 44 - protein, 10 aa, Artificial sequence
  Synthesized
GRKARRQRRR

SEQ ID NO: 45 - protein, 9 aa, Artificial sequence
  Synthesized
RKARRQRRR

SEQ ID NO: 46 - protein, 10 aa, Artificial sequence
  Synthesized
GRKKARQRRR

SEQ ID NO: 47 - protein, 9 aa, Artificial sequence
  Synthesized
RKKARQRRR

SEQ ID NO: 48 - protein, 10 aa, Artificial sequence
  Synthesized
GRKKRRQARR

SEQ ID NO: 49 - protein, 9 aa, Artificial sequence
  Synthesized
RKKRRQARR

SEQ ID NO: 50 - protein, 10 aa, Artificial sequence
  Synthesized
GRKKRRQRAR

SEQ ID NO: 51 - protein, 9 aa, Artificial sequence
  Synthesized
RKKRRQRAR

SEQ ID NO: 52 - protein, 11 aa, Artificial sequence
  Synthesized
RRPRRPRRPRR

SEQ ID NO: 53 - protein, 11 aa, Artificial sequence
  Synthesized
RRARRARRARR

SEQ ID NO: 54 - protein, 10 aa, Artificial sequence
  Synthesized
RRRARRRARR

SEQ ID NO: 55 - protein, 10 aa, Artificial sequence
  Synthesized
RRRPRRRPRR

SEQ ID NO: 56 - protein, 8 aa, Artificial sequence
  Synthesized
RRPRRPRR

SEQ ID NO: 57 - protein, 8 aa, Artificial sequence
  Synthesized
RRARRARR

SEQ ID NO: 58 - protein, 20 aa, Artificial sequence
  Synthesized
YGRKKRRQRRRKLSSIESDV

SEQ ID NO: 59 - protein, 20 aa, Artificial sequence
  Synthesized
  MISC_FEATURE (1)..(16): D-amino acids
YGRKKRRQRRRKLSSIESDV

SEQ ID NO: 60 - protein, 20 aa, Artificial sequence
  Synthesized
  MISC_FEATURE (1)..(14): D-amino acids
YGRKKRRQRRRKLSSIESDV

SEQ ID NO: 61 - protein, 20 aa, Artificial sequence
  Synthesized
  MISC_FEATURE (1)..(13): D-amino acids
YGRKKRRQRRRKLSSIESDV

SEQ ID NO: 62 - protein, 20 aa, Artificial sequence
  Synthesized
  MISC_FEATURE (1)..(15): D-amino acids
YGRKKRRQRRRKLSSIETDV

SEQ ID NO: 63 - protein, 20 aa, Artificial sequence
  Synthesized
  MISC_FEATURE (1)..(16): D-amino acids
YGRKKRRQRRRKLSSIETDV

SEQ ID NO: 64 - protein, 20 aa, Artificial sequence
  Synthesized
  MISC_FEATURE (1)..(14): D-amino acids
YGRKKRRQRRRKLSSIETDV

SEQ ID NO: 65 - protein, 19 aa, Artificial sequence
  Synthesized
  MISC_FEATURE (1)..(14): D-amino acids
YGRKKRRQRRRKSSIETDV

SEQ ID NO: 66 - protein, 18 aa, Artificial sequence
  Synthesized
  MISC_FEATURE (1)..(13): D-amino acids
YGRKKRRQRRRKSIETDV

SEQ ID NO: 67 - protein, 17 aa, Artificial sequence
  Synthesized
  MISC_FEATURE (1)..(12): D-amino acids
YGRKKRRQRRRKIETDV

SEQ ID NO: 68 - protein, 16 aa, Artificial sequence
  Synthesized
  MISC_FEATURE (1)..(11): D-amino acids
YGRKKRRQRRRIESDV

SEQ ID NO: 69 - protein, 16 aa, Artificial sequence
  Synthesized
  MISC_FEATURE (1)..(11): D-amino acids
YGRKKRRQRRRIETDV

SEQ ID NO: 70 - protein, 9 aa, Artificial sequence
  Synthesized
  MISC_FEATURE (1)..(2): D-amino acids
KLSSIESDV

SEQ ID NO: 71 - protein, 8 aa, Artificial sequence
  Synthesized
  MISC_FEATURE (1)..(1): D-amino acid
LSSIESDV

SEQ ID NO: 72 - protein, 8 aa, Artificial sequence
  Synthesized
LSSIESDV

SEQ ID NO: 73 - protein, 7 aa, Artificial sequence
  Synthesized
SSIESDV

SEQ ID NO: 74 - protein, 6 aa, Artificial sequence
  Synthesized
SIESDV

SEQ ID NO: 75 - protein, 9 aa, Artificial sequence
  Synthesized
  MISC_FEATURE (1)..(2): D-amino acids
KLSSIETDV

SEQ ID NO: 76 - protein, 8 aa, Artificial sequence
  Synthesized
  MISC_FEATURE (1)..(1): D-amino acid
LSSIETDV

SEQ ID NO: 77 - protein, 8 aa, Artificial sequence
  Synthesized
LSSIETDV

SEQ ID NO: 78 - protein, 7 aa, Artificial sequence
  Synthesized
SSIETDV

SEQ ID NO: 79 - protein, 6 aa, Artificial sequence
  Synthesized
SIETDV

SEQ ID NO: 80 - protein, 20 aa, Artificial sequence
  Synthesized
  MISC_FEATURE (1)..(11): D-amino acids
YGRKKRRQRRRKLSSIESDV

SEQ ID NO: 81 - protein, 20 aa, Artificial sequence
  Synthesized
  MISC_FEATURE (1)..(20): D-amino acids
YGRKKRRQRRRKLSSIESDV

SEQ ID NO: 82 - protein, 15 aa, Artificial sequence
  Synthesized
RRQRRRKLSSIESDV

SEQ ID NO: 83 - protein, 14 aa, Artificial sequence
  Synthesized
RQRRRKLSSIESDV

SEQ ID NO: 84 - protein, 13 aa, Artificial sequence
  Synthesized
QRRRKLSSIESDV

SEQ ID NO: 85 - protein, 11 aa, Artificial sequence
  Synthesized
RRKLSSIESDV

SEQ ID NO: 86 - protein, 10 aa, Artificial sequence
  Synthesized
RKLSSIESDV

SEQ ID NO: 87 - protein, 8 aa, Artificial sequence
  Synthesized
LSSIESDV